(19) United States
(12) Patent Application Publication
(10) Pub. No.: US 2026/0265374 A1
(43) Pub. Date: Sep. 10, 2026

(54) FUSION PROTEIN AND MEDICAL USE THEREOF

(71) Applicants: JW THERAPEUTICS R & D (SHANGHAI) CO., LTD., Shanghai (CN); JW THERAPEUTICS (SHANGHAI) CO., LTD., Shanghai (CN)

(72) Inventors: Haihua GU, Shanghai (CN); Jun GUO, Shanghai (CN); Shaun CORDOBA, Shanghai (CN)

(21) Appl. No.: 19/491,301

(22) PCT Filed: Jun. 7, 2024
(86) PCT No.: PCT/CN2024/098107

§ 371 Date: Dec. 8, 2025

(30) Foreign Application Priority Data
Jun. 9, 2023 (WO) PCT/CN2023/099418
May 9, 2024 (WO) PCT/CN2024/091948

(51) Int. Cl. C07K 16/28; A61K 38/00; A61K 40/11; A61K 40/15; A61K 40/31; A61K 40/42; C07K 14/705; C12N 5/0783

(52) U.S. Cl. CPC C07K 16/2803; A61K 40/11; A61K 40/15; A61K 40/31; A61K 40/4211; C07K 14/7051; C07K 14/70578; C12N 5/0636; A61K 38/00; C07K 2319/02; C07K 2319/03; C12N 2510/00

(57) ABSTRACT

The present disclosure provides fusion proteins comprising modified spacer domains that increases the antigen binding affinity, antibodies directing against CD19, and their use in the treatment of disease thereof.

### CROSS-REFERENCE TO RELATED APPLICATIONS

This application is based on international patent applications with the application number PCT/CN2023/099418 and PCT/CN2024/091948, and claims priority from said international patent applications, the entire content of which is hereby incorporated by reference into this application.

### FIELD OF THE INVENTION

The instant disclosure relates generally to fusion proteins comprising modified hinge domain and method of use thereof.

### BACKGROUND

Chimeric antigen receptor (CAR) T cells have shown remarkable response rates in patients with specific B cell leukemia or lymphoma subtypes, and encouraging results have also been observed in patients with multiple myeloma. A CAR construct usually comprises an extracellular antigen-binding domain, a spacer, a transmembrane domain and an intracellular signaling domain. The spacer located between the extracellular antigen-binding domain and the transmembrane domain plays a critical role in mediating antitumor activity. There is a need for CARs with improved spacer domain.

### BRIEF SUMMARY

The present disclosure provides novel fusion proteins comprising modified spacer domain with advantageous properties for cell therapy. Specifically, the disclosure relates to CARs having modified spacers that allow the CARs to retain the ability of stabilization, maintain the conformation of the antigen-binding domain in CAR, and/or position the antigen-binding domain at a distance from a target epitope such that the CAR can effectively interact with the target cell.

In a first aspect, the disclosure provides a fusion protein comprising a) an extracellular antigen-binding domain; b) a spacer that consists of the sequence of SEQ ID NO: 28 or a functional equivalent thereof; c) a transmembrane domain; and d) an intracellular signaling domain.

Also provided herein is a fusion protein, wherein the spacer consists of the amino acid sequence of SEQ ID NO: 31 (EX₁KSCDTPPPX₂PX₃CP); wherein X₁ is S or P; X₂ is C or S; and X₃ is R or P. In one aspect, the spacer consists of the amino acid sequence of SEQ ID NO: 31 (EX₁KSCDTPPPX₂PX₃CP); wherein X₁ is S; X₂ is C or S; and X₃ is R or P. In one aspect, the spacer consists of the amino acid sequence of SEQ ID NO: 28.

Also provided herein is a fusion protein, wherein the extracellular antigen-binding domain comprises an antibody comprising a heavy chain variable region (VH) and a light chain variable region (VL). In one aspect, the antibody is capable of binding to the same epitope on CD19 as a reference antibody that comprises the amino acid sequence of SEQ ID NO: 21 or SEQ ID NO: 25. In one aspect, the VH comprises a HCDR1, a HCDR2 and a HCDR3 comprising the amino acid sequences of SEQ ID NO: 5, SEQ ID NO: 6 and SEQ ID NO: 7, respectively; the VL comprises a LCDR1, a LCDR2 and a LCDR3 comprising the amino acid sequences of SEQ ID NO: 8, SEQ ID NO: 9 and SEQ ID NO: 10, respectively. In one aspect, the VH comprises a HCDR1, a HCDR2 and a HCDR3 comprising the amino acid sequences of SEQ ID NO: 13, SEQ ID NO: 14 and SEQ ID NO: 15, respectively; the VL comprises a LCDR1, a LCDR2 and a LCDR3 comprising the amino acid sequences of SEQ ID NO: 16, SEQ ID NO: 17 and SEQ ID NO: 18, respectively.

Also provided herein is a fusion protein, wherein the VH comprises the amino acid sequence of SEQ ID NO: 1; the VL comprises the amino acid sequence of SEQ ID NO: 2, or the amino acid sequence having at least 90% sequence identity to SEQ ID NO: 2, wherein the amino acids of the VL at positions 39 and 80 are R and P, respectively. In one aspect, the VL comprises an amino acid sequence having at least 95% identity to SEQ ID NO: 2, wherein the amino acids of the VL at positions 39 and 80 are R and P, respectively. In one aspect, the amino acids of the VL at positions 100 and 103 are S and R, respectively. In one aspect, the amino acids of the VL at positions 39, 80, 100 and 103 are R, P, S and R, respectively. In one aspect, the VH comprises the amino acid sequence of SEQ ID NO: 1; the VL comprises the amino acid sequence of SEQ ID NO: 3 or SEQ ID NO: 4.

Also provided herein is a fusion protein, wherein the antibody is an scFv. In one aspect, the VH is fused by its N-terminus via a flexible polypeptide linker to the C-terminus of the VL. In one aspect, the flexible polypeptide linker comprises the amino acid sequence of SEQ ID NO: 19 or SEQ ID NO: 20. In one aspect, the scFv comprises the amino acid sequence of SEQ ID NO: 21, SEQ ID NO: 22, SEQ ID NO: 23 or SEQ ID NO: 24. In one aspect, the scFv comprises the amino acid sequence of SEQ ID NO: 21, SEQ ID NO: 22 or SEQ ID NO: 23. In one aspect, the scFv comprises the amino acid sequence of SEQ ID NO: 22.

Also provided herein is a fusion protein, wherein the transmembrane domain comprises a transmembrane domain of CD8α, CD4, CD28, CD137, CD80, CD86, CD152 or PD1. In one aspect, the transmembrane domain comprises a transmembrane domain of CD8α, CD4 or CD28. In one aspect, the transmembrane domain comprises the amino acid sequence of SEQ ID NO: 32.

Also provided herein is a fusion protein, wherein the intracellular signaling domain comprises a co-stimulatory signaling domain and/or a primary intracellular signaling domain. In one aspect, the co-stimulatory signaling domain is fused by its C-terminus to the N-terminus of the primary intracellular signaling domain. In one aspect, the co-stimulatory signaling domain is selected from the group consisting of a signaling domain of CD27, CD28, CD137, OX40, CD30, CD40, CD3, HVEM, ICOS, Myd88, LFA-1, ICOS, CD2, CD7, NKG2C, B7-H3, Ligands of CD83 and a combination thereof. In one aspect, the co-stimulatory signaling domain is a signaling domain of CD28 or CD137. In one aspect, the co-stimulatory signaling domain is a signaling domain of CD137. In one aspect, the primary intracellular signaling domain comprises a cytoplasmic signaling domain of CD3λ. In one aspect, the co-stimulatory signaling domain is a signaling domain of CD28 or CD137; the primary intracellular signaling domain comprises a cytoplasmic signaling domain of CD3λ. In one aspect, the co-stimulatory signaling domain is a signaling domain of CD137; the primary intracellular signaling domain comprises a cytoplasmic signaling domain of CD3λ.

Also provided herein is a fusion protein, wherein the fusion protein is a CAR that comprises, from its N to C terminus in order: the extracellular antigen-binding domain, the spacer, the transmembrane domain and the intracellular signaling domain. In one aspect, the extracellular antigen-binding domain comprises the amino acid sequence of SEQ ID NO: 21, SEQ ID NO: 22 or SEQ ID NO: 23, the spacer consists of the amino acid sequence of SEQ ID NO: 28. In one aspect, the CAR comprises the amino acid sequence of SEQ ID NO: 35, SEQ ID NO: 36 or SEQ ID NO: 37. In one aspect, the CAR comprises the amino acid sequence of SEQ ID NO: 35 or SEQ ID NO: 36.

A second aspect of the disclosure provides an antibody that binds to CD19 comprising a VH and a VL, wherein the VH comprises the sequence of SEQ ID NO: 1; the VL comprises an amino acid sequence having at least 90% identity to SEQ ID NO: 2, wherein the amino acids of the VL at positions 39 and 80 are R and P, respectively. In one aspect, the VL comprises an amino acid sequence having at least 95% identity to SEQ ID NO: 2, wherein the amino acids of the VL at positions 39 and 80 are R and P, respectively. In one aspect, the amino acids of the VL at positions 100 and 103 are S and R, respectively. In one aspect, the amino acids of the VL at positions 39, 80, 100 and 103 are R, P, S and R, respectively. In one aspect, the VH comprises the amino acid sequence of SEQ ID NO: 1; the VL comprises the amino acid sequence of SEQ ID NO: 3 or SEQ ID NO: 4.

Also provided herein is an antibody, wherein the antibody is an scFv. In one aspect, the VH is fused by its N-terminus via a flexible polypeptide linker to the C-terminus of the VL. In one aspect, the flexible polypeptide linker comprises the amino acid sequence of SEQ ID NO: 19 or SEQ ID NO: 20. In one aspect, the scFv comprises the amino acid sequence of SEQ ID NO: 21, SEQ ID NO: 22, SEQ ID NO: 23 or SEQ ID NO: 24. In one aspect, the scFv comprises the amino acid sequence of SEQ ID NO: 21, SEQ ID NO: 22 or SEQ ID NO: 23. In one aspect, the scFv comprises the amino acid sequence of SEQ ID NO: 22.

The disclosure further provides a complex, comprising the antibody according to the disclosure as described herein and a heterologous molecule or moiety. In one aspect, the heterologous molecule or moiety is covalently bound to the antibody. In one aspect, the complex is a fusion protein comprising, from its N to C terminus in order: an extracellular antigen binding domain comprising the antibody, a transmembrane domain and an intracellular signaling domain.

The disclosure further provides an isolated nucleic acid encoding the fusion protein or the antibody according to the disclosure as described herein.

The disclosure further provides a vector comprising the nucleic acid according to the disclosure as described herein. In one aspect, the vector is a viral vector. In one aspect, the viral vector is a retroviral, lentiviral, adenoviral, or adeno-associated viral vector.

The disclosure further provides a cell comprising the fusion protein, the antibody, the complex, the isolated nucleic acid, or the vector according to the disclosure as described herein. In one aspect, the cell is a lymphocyte. In one aspect, the cell is a NK cell or a T cell.

The disclosure further provides a pharmaceutical formulation comprising a fusion protein, the antibody, the complex or the cell according to the disclosure as described herein and a pharmaceutically acceptable carrier.

The disclosure further provides a polypeptide consisting of the amino acid sequence of SEQ ID NO: 31 (EX₁KSCDTPPPX₂PX₃CP), wherein X₁ is S or P; X₂ is C or S; and X₃ is R or P. In one aspect, the polypeptide is comprised in a fusion protein. In one aspect, the polypeptide consists of the amino acid sequence of SEQ ID NO: 31 (EX₁KSCDTPPPX₂PX₃CP); wherein X₁ is S; X₂ is C or S; and X₃ is R or P. In one aspect, the polypeptide consists of the amino acid sequence of SEQ ID NO: 28.

The disclosure further provides a method of treating a cancer or an autoimmune disease in a subject, comprising administering the fusion protein, the antibody, the complex, the cell or the pharmaceutical formulation according to the disclosure as described herein to the subject. Also provided is the use of the fusion protein, the antibody, the complex, the cell or the pharmaceutical formulation according to the disclosure as described herein in the manufacture of a medicament for the treatment of a cancer or an autoimmune disease. The disclosure further provides the fusion protein, the antibody, the complex, the cell or the pharmaceutical formulation according to the disclosure as described herein for use as a medicament. In one aspect, the cancer is selected from the group consisting of leukemia, lymphoma, lung cancer, melanoma, breast cancer, prostate cancer, colon cancer, renal cell carcinoma, ovarian cancer, neuroblastoma and rhabdomyosarcoma. In one aspect, the cancer is leukemia or lymphoma. In one aspect, the autoimmune disease is selected from the group consisting of systemic lupus erythematosus, lupus nephritis, multiple sclerosis, rheumatoid arthritis, Sjogren's syndrome, idiopathic thrombocytopenia purpura, Type 1 diabetes, Pemphigus vulgaris, Neuromyelitis optica, ANCA vasculitis, and Myasthenia gravis. In one aspect, the autoimmune disease is systemic lupus erythematosus or lupus nephritis.

### BRIEF DESCRIPTION OF THE DRAWINGS

FIG. 1A to 1C show the association constant (Kₐ), association rate (Kₒₙ) and dissociation rate (K_{off}) of FMC63-scFv, scFv-1, scFv-2 and scFv-3, respectively.

FIG. 2 shows the binding affinity to CD19 of CAR-W and CAR-1 to 12 on T cells.

FIG. 3 relates to the tonic signaling of CAR-W and CAR-1 to 12.

In FIG. 4A to 4C the activities of CARs comprising spacer S-3 is shown. FIG. 4A shows the degree of CD19 expression of different target cells. The killing efficiency of the CARs is shown in Figure. 4B. FIG. 4C shows the CD69 expression on T cells induced by CARs.

FIG. 5A to 5D relates to the T cell activation and expansion post target cell stimulation. FIGS. 5A, 5B, and 5C show the IFN-γ, TNF-α, and IL-2 secreted by T cell transduced with CARs. FIG. 5D shows the proliferation of T cell 7 days after the initial simulation.

FIG. 6 shows the CAR-T cell killing of Raji-CD19Lo target cells at day 3 of co-culture assay (E:T=1:1, 1:2, 1:8).

### DETAILED DESCRIPTION

### I. Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “a module” means one module or more than one module.

The terms “comprising”, “comprises” as used herein are synonymous with “including”, “includes” or “containing”, “contains”, and are inclusive or open-ended and do not exclude additional, non-recited members, elements or method steps. The terms “comprising”, “comprises” and the like also include the term “consisting of”.

The term “antibody” herein is used in the broadest sense and encompasses various antibody structures, including but not limited to monoclonal antibodies, polyclonal antibodies, multispecific antibodies (e.g. bispecific antibodies), intact antibody and antibody fragments so long as they exhibit the desired antigen-binding activity.

The terms “full-length antibody,” “intact antibody,” and “whole antibody” are used herein interchangeably to refer to an antibody having a structure substantially similar to a native antibody structure. For example, an intact antibody of IgG class comprises two light chains and two heavy chains that are disulfide-bonded. From N- to C-terminus, each heavy chain has a variable domain (VH), followed by three constant domains (CH1, CH2, and CH3), also called a heavy chain constant region. Similarly, from N- to C-terminus, each light chain has a variable domain (VL), followed by a constant light (CL) domain, also called a light chain constant region. The heavy chain of an antibody may be assigned to one of five types, called IgA, IgD, IgE, IgG, or IgM, some of which may be further divided into subtypes, e.g. IgG1, IgG2, IgG3, IgG4, IgAQ1 and IgA2. The light chain of an antibody may be assigned to one of two types, called kappa (κ) and lambda (λ), based on the amino acid sequence of its constant domain.

The “antibody fragment” refers to a molecule other than an intact antibody that comprises a portion of an intact antibody that binds the antigen. Examples of antibody fragments include but are not limited to Fv, Fab, Fab′, Fab′-SH, F(ab′)₂, diabodies, linear antibodies, single-chain antibody molecules (e.g. scFv and scFab), single-domain antibodies, and multispecific antibodies formed from antibody fragments.

A “Single-chain Fv” or “scFv” is a fusion protein of the variable regions of the light (VL) and heavy chain (VH), connected with a short linker peptide of about ten to 25 amino acids. The linker is usually flexible, and can either connect the N-terminus of the VH with the C-terminus of the VL, or vice versa. This protein retains the specificity of the original antibody, despite removal of the constant regions and the introduction of the linker.

The term “antigen binding domain” refers to the part of an antibody that comprises the area which binds to and is complementary to part or all of an antigen. An antigen binding domain may be provided by, for example, one or more antibody variable domains (also called antibody variable regions). In preferred aspects, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and complementarity determining regions (CDRs). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

The terms “complementarity determining region” and “CDR” are known to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3). “Framework regions” and “FR” refer to the non-CDR portions of the variable regions of the heavy and light chains. Table 1, below, lists exemplary position boundaries of HCDR1, HCDR2, HCDR3, LCDR1, LCDR2 and LCDR3 as identified by Kabat, Chothia, AbM, IMGT and Contact schemes, respectively. Unless otherwise indicated, the amino acid sequences of CDRs shown in the present disclosure are determined according to Kabat.

TABLE 1
the CDR regions according to various numbering systems

| CDR | Kabat | AbM | Chothia | IMGT | Contact |
|---|---|---|---|---|---|
| HCDR1 | 31-35B | 26-35 | 26-32 | 27-38 | 30-35 |
| HCDR2 | 50-65 | 50-58 | 52-56 | 56-65 | 47-58 |
| HCDR3 | 95-102 | 95-102 | 95-102 | 105-117 | 93-101 |
| LCDR1 | 24-34 | 24-34 | 24-34 | 27-38 | 30-36 |
| LCDR2 | 50-56 | 50-56 | 50-56 | 56-65 | 46-55 |
| LCDR3 | 89-97 | 89-97 | 89-97 | 105-117 | 89-96 |

The term “humanized” antibody refers to a chimeric antibody comprising amino acid residues from non-human CDRs and amino acid residues from human frameworks. In certain embodiments, a humanized antibody will comprise at least one, and typically two, variable domains, in which all or substantially all of the CDRs correspond to those of a non-human antibody, and all or substantially all of the FRs correspond to those of a human antibody.

The term “human” antibody refers to an antibody that possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “specifically binds” or “bind”, which can be used interchangeably, as used herein means the ability of a protein, under specific binding conditions, to bind to a target protein such that its affinity or avidity is at least 5 times as great, but optionally at least 10, 20, 30, 40, 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to a non-target molecule due to similarity in structural conformation between the target and non-target (e.g., paralogs or orthologs). One skilled artisan recognizes that specific binding to a molecule having the same function in a different species of animal or to a non-target molecule having a substantially similar epitope as the target molecule is possible and does not detract from the specificity of binding which is determined relative to a statistically valid collection of unique non-targets. Thus, a polypeptide may specifically bind to more than one distinct species of target molecule due to cross-reactivity.

The term “epitope” refers to the moieties of an antigen that specifically interact with an antibody. Such moieties, referred to herein as epitopic determinants, typically comprise, or are part of, elements such as amino acid side chains or sugar side chains. An epitopic determinate can be defined, e.g. by methods known in the art, e.g. by crystallography or by hydrogen-deuterium exchange. At least one or some of the moieties on the antibody molecule that specifically interact with an epitopic determinant are typically located in a CDR(s). Typically, an epitope has a specific three-dimensional structural characteristic and/or a specific charge characteristic. Some epitopes are linear epitopes while others are conformational epitopes.

The term “hinge” refers to the part of an antibody heavy chain polypeptide that joins in a wild-type antibody heavy chain the CH1 domain and the CH2 domain. In the present disclosure, the hinges contained in fusion proteins comprise naturally occurring or modified sequences.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “fragment” of a polypeptide (e.g. a hinge) refers to an amino acid sequence of a polypeptide that is shorter than the naturally occurring sequence, N- and/or C-terminally deleted or any part of the polypeptide deleted in comparison to the naturally occurring polypeptide. Thus, a fragment does not necessarily need to have only N- and/or C-terminal amino acids deleted. A polypeptide in which internal amino acids have been deleted with respect to the naturally occurring sequence is also considered a fragment. The term “C-terminus fragment” refers to a fragment having the deletion of N-terminal amino acids of the naturally occurring polypeptide only.

The term “functional equivalent” refers to a polypeptide that retains polypeptide function. Accordingly, in some aspects, a functional equivalent of a hinge, retains the ability to stabilize the CAR molecule, maintain the conformation and valency of the antigen-binding domain (e.g. an scFv) in a CAR and/or position the antigen-binding domain at a distance from a target epitope such that the CAR can effectively interact with the target cell.

The term “fusion protein” refers to a hybrid (e.g., chimeric, recombinant) polypeptide which comprises protein domains from at least two different proteins.

The term “Chimeric Antigen Receptor” or alternatively a “CAR” refers to a fusion protein engineered to contain two or more naturally occurring or engineered amino acid sequences linked together in a way that does not occur naturally in a host cell, which can function as a receptor when present on a surface of a cell. CARs of the present disclosure include an extracellular portion comprising an antigen-binding domain (e.g. an scFv) linked to a spacer sequence, a transmembrane domain and one or more intracellular signaling domains (optionally containing co-stimulatory domain(s)).

The term “extracellular domain” or “ectodomain” which can be used interchangeably, as used herein refers to the region of a membrane protein, such as a transmembrane protein, that lies outside the vesicular membrane. Ectodomains often comprise binding domains that specifically bind to ligands or cell surface receptors, such as via a binding domain that specifically binds to the ligand or cell surface receptor. The term “extracellular antigen-binding domain” refers to an extracellular domain or a part of extracellular domain that is capable of specifically binding to an antigen.

The term “endodomain” or “intracellular domain,” or “cytoplasmic domain” which can be used interchangeably, as used herein refers to the region found in some membrane proteins, such as transmembrane proteins, which extends into the interior space defined by the cell surface membrane. In some cells, the endodomain interacts with intracellular constituents and can play a role in signal transduction and thus, in some cases, can be an intracellular signaling domain.

The term “transmembrane domain” as used herein means a domain found in a membrane protein that substantially or completely spans a lipid bilayer such as those lipid bilayers found in a biological membrane such as a mammalian cell, or in an artificial construct such as a liposome. A transmembrane protein can pass through both layers of the lipid bilayer once or multiple times.

The term “spacer” as used herein refers to a polypeptide sequence which is capable of covalently linking together two spaced moieties: an antigen-binding domain, and the transmembrane domain of the fusion protein (e.g. a CAR).

The term “flexible polypeptide linker” as used herein refers to a peptide which joins the other peptides to form a functional protein. In the context of an scFv, the flexible polypeptide linker refers to a peptide linker which joins the VH and VL to form an scFv specifically binding to an antigen.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently different genetically to interact antigenically.

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system.

The terms “polypeptide”, “peptide” and “protein” are used interchangeably, and refer to a compound comprising amino acid residues covalently linked by peptide bonds. A polypeptide must contain at least two amino acids, and no limitation is placed on the maximum number of amino acids that can comprise a peptide's sequence. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide of the disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure.

An “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that is different from its natural chromosomal location. “Isolated polynucleotide (or nucleic acid) encoding a fusion protein” refers to one or more polynucleotide molecules encoding the fusion protein, including such polynucleotide molecule(s) in a single vector or separate vectors, and such polynucleotide molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that has the same function or biological activity as screened or selected for in the originally transformed cell are included herein. A host cell is any type of cellular system that can be used to generate the antibodies of the present invention. Host cells include cultured cells, e.g. mammalian cultured cells, such as HEK cells, CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. In one aspect, the host cell of the invention is a eukaryotic cell, particularly a mammalian cell. In one aspect, the host cell is not a cell within a human body.

The term “subject” or “individual” is intended to include living organisms in which an immune response can be elicited (e.g., mammals, human).

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “pharmaceutical composition” refers to a preparation which is in such form as to permit the biological activity of an active ingredient contained therein to be effective, and which contains no additional components which are unacceptably toxic to a subject to which the preparation would be administered. A pharmaceutical composition usually comprises one or more pharmaceutically acceptable carrier(s). A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

An “effective amount” of an agent, e.g., a pharmaceutical composition, refers to an amount effective, at dosages and for periods of time necessary, to achieve the desired therapeutic or prophylactic result.

### II. Fusion Protein

The present disclosure provides a novel fusion protein with advantageous properties such as producibility, stability, binding affinity, biological activity, targeting efficiency, reduced toxicity, an extended dosage range that can be given to a patient (e.g. contained in a cell) and thereby a possibly enhanced efficacy. The novel fusion protein comprises a spacer that improves antigen binding, CAR stability and ability to kill target cells that express low density ligands.

Herein provided is a fusion protein comprising a) an extracellular antigen-binding domain; b) a spacer that consists of the amino acid sequence of SEQ ID NO: 28 or a functional equivalent thereof; c) a transmembrane domain; and d) an intracellular signaling domain. In some embodiments, the fusion protein is a CAR.

### 2.1 Spacer

In one aspect, provided is the fusion protein as defined herein before, wherein the fusion protein comprising the spacer that consists of the functional equivalent of SEQ ID NO: 28 exhibits no more than 10%, 9%, 8%, 7%, 6%, 5%, 4%, 3%, 2%, or 1% difference in the value of CD19 binding compared with the fusion protein comprising the spacer that consists of SEQ ID NO: 28.

In one aspect, the spacer includes at least a portion of an immunoglobulin constant region or variant or modified version thereof, such as a hinge region. In one aspect, the spacer includes at least a portion of IgG hinge. In one aspect, the spacer can be a chimeric polypeptide containing one or more fragments of hinges derived from IgG1, IgG2, IgG3 and/or IgG4. In one aspect, the spacer can be a chimeric polypeptide containing one or more fragments of hinges derived from IgG3. In one aspect, the spacer consists of an amino acid sequence that is 11, 12, 13, 14, 15, 16, 17, 18, 19 or 20 amino acid residues in length. In some embodiment, the spacer consists of an amino acid sequence that is 13-17 amino acid residues in length. In a preferred embodiment, the spacer consists of an amino acid sequence that is 15 amino acid residues in length. In one aspect, the spacer consists of an amino acid sequence that is at least about 80%, 85%, 90% or 95% identical to the amino acid sequence of SEQ ID NO: 28.

In one aspect, the spacer consists of the amino acid sequence of SEQ ID NO: 31 (EX₁KSCDTPPPX₂PX₃CP); wherein X₁ is S or P; X₂ is C or S; and X₃ is R or P. In one aspect, X₁ is S; X₂ is C or S; and X₃ is R or P. In one aspect, X₁ is P; X₂ is C or S; and X₃ is R or P. In one aspect, X₁ is S or P; X₂ is C; and X₃ is R or P. In one aspect, X₁ is S or P; X₂ is S; and X₃ is R or P. In one aspect, X₁ is S or P; X₂ is C or S; and X₃ is R. In one aspect, X₁ is S or P; X₂ is C or S; and X₃ is P. In one aspect, X₁ is S; X₂ is C; and X₃ is R or P. In one aspect, X₁ is S; X₂ is S; and X₃ is R or P. In one aspect, X₁ is S; X₂ is C; and X₃ is R. In one aspect, X₁ is S; X₂ is C; and X₃ is P. In one aspect, X₁ is S; X₂ is S; and X₃ is R. In one aspect, X₁ is S; X₂ is S; and X₃ is P. In a particular embodiment, the spacer consists of the amino acid sequence of SEQ ID NO: 28.

### 2.2 Extracellular Antigen-Binding Domain

In one aspect, provided is the fusion protein as defined herein before, wherein the extracellular antigen-binding domain comprises one or more antibody. In one aspect, the antibody is Fv, Fab, Fab′, Fab′-SH, F(ab′)₂, diabodies, linear antibodies, single-chain antibody molecules (e.g. scFv and scFab), single-domain antibodies, or multispecific antibodies formed from antibody fragments. In a particular embodiment, the extracellular antigen-binding domain comprises an scFv.

In one aspect, the extracellular antigen binding domain is capable of binding to a tumor antigen or a pathogen antigen. In one aspect, the tumor antigen is a tumor-specific antigen or a tumor-associated antigen (TAA). Various TAAs are known, some of which are shown in the following Table 2. The antigen-binding domain used in the present disclosure is a domain which is capable of binding a TAA as indicated therein.

TABLE 2
Type of the TAA

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |

In one aspect, the tumor antigen is CD19. In one aspect, the fusion protein is capable of binding to the same epitope on CD19 as any anti-CD19 antibody in the art (e.g. FMC63, SC25C1). In one aspect, the extracellular antigen-binding domain of the fusion protein comprises an scFv that is capable of binding to the same epitope on CD19 as a reference antibody that comprises the amino acid sequence of SEQ ID NO: 21 or SEQ ID NO: 25. In one aspect, the scFv comprises a VH and a VL, the VH comprises a HCDR1, a HCDR2 and a HCDR3 comprising the amino acid sequences of SEQ ID NO: 5, SEQ ID NO: 6 and SEQ ID NO: 7, respectively; the VL comprises a LCDR1, a LCDR2 and a LCDR3 comprising the amino acid sequences of SEQ ID NO: 8, SEQ ID NO: 9 and SEQ ID NO: 10, respectively. In one aspect, the scFv comprises a VH and a VL, the VH comprises a HCDR1, a HCDR2 and a HCDR3 comprising the amino acid sequences of SEQ ID NO: 13, SEQ ID NO: 14 and SEQ ID NO: 15, respectively; the VL comprises a LCDR1, a LCDR2 and a LCDR3 comprising the amino acid sequences of SEQ ID NO: 16, SEQ ID NO: 17 and SEQ ID NO: 18, respectively.

In one aspect, the scFv comprises a VH and a VL, wherein the VH comprises the amino acid sequence of SEQ ID NO: 1; the VL comprises the amino acid sequence of SEQ ID NO: 2. In one aspect, the VH comprises the amino acid sequence having at least 90%, 91%, 92%, 93%, 94%, 95%, 96%, 97% or 98% sequence identity to SEQ ID NO: 1. In one aspect, the VL comprises the amino acid sequence having at least 90%, 91%, 92%, 93%, 94%, 95%, 96%, 97% or 98% sequence identity to SEQ ID NO: 2. In one aspect, the VL comprises the amino acid sequence having at least 90%, 91%, 92%, 93%, 94%, 95%, 96%, 97% or 98% sequence identity to SEQ ID NO: 2, wherein the amino acids of the VL at positions 39 and 80 are R and P, respectively. In one aspect, the VL comprises or consists of the amino acid sequence having at least 90%, 91%, 92%, 93%, 94%, 95% or 96% sequence identity to SEQ ID NO: 2, wherein the amino acids of the VL at positions 39, 80, 100 and 103 are R, P, S and R, respectively. In a particular embodiment, the VH comprises or consists of the amino acid sequence of SEQ ID NO: 1; the VL comprises the amino acid sequence of SEQ ID NO: 3 or SEQ ID NO: 4.

In one aspect, the scFv comprises a VH and a VL of a humanized antibody or a human antibody. Exemplary scFvs are described in patents No WO2010095031, WO2014153270, WO2017015783, CN107383196A, CN111848801A, WO2018200496 or WO2022105811.

In one aspect, the VH and the VL are connected via a flexible polypeptide linker. In one aspect, the VH is fused by its N-terminus via a flexible polypeptide linker to the C-terminus of the VL (L-H). In one aspect, the VL is fused by its N-terminus via a flexible polypeptide linker to the C-terminus of the VH (H-L). In one aspect, the flexible polypeptide linker is (G4S)ₙ, (SG₄)ₙ or G₄(SG₄)ₙ, wherein “n” is 1, 2, 3, 4, 5, 6, 7 or 8, in particular 3. In one aspect, the flexible polypeptide linker comprises the amino acid sequence of SEQ ID NO: 19 or SEQ ID NO: 20. In one aspect, the scFv comprises the amino acid sequence having at least 90%, 91%, 92%, 93%, 94%, 95%, 96%, 97% or 98% sequence identity to SEQ ID NO: 21, SEQ ID NO: 22, SEQ ID NO: 23, SEQ ID NO: 24 or SEQ ID NO: 25. In one aspect, the scFv comprises the amino acid sequence of SEQ ID NO: 21, SEQ ID NO: 22, SEQ ID NO: 23, SEQ ID NO: 24 or SEQ ID NO: 25. Table 3 provides the SEQ ID NOs: of exemplary scFvs.

TABLE 3
SEQ ID NOs of the exemplary scFvs

| No | HCDR1 | HCDR2 | HCDR3 | LCDR1 | LCDR2 | LCDR3 | VH | VL | Linker | Order | scFv |
|---|---|---|---|---|---|---|---|---|---|---|---|
| FMC63 | 5 | 6 | 7 | 8 | 9 | 10 | 1 | 2 | 19 | L-H | 21 |
| scFv | | | | | | | | | | | |
| scFv-1 | 5 | 6 | 7 | 8 | 9 | 10 | 1 | 3 | 19 | L-H | 22 |
| scFv-2 | 5 | 6 | 7 | 8 | 9 | 10 | 1 | 4 | 19 | L-H | 23 |
| scFv-3 | 5 | 6 | 7 | 8 | 9 | 10 | 1 | 2 | 19 | H-L | 24 |
| SJ25C1 | 13 | 14 | 15 | 16 | 17 | 18 | 11 | 12 | 20 | L-H | 25 |
| scFv | | | | | | | | | | | |

### 2.3 Transmembrane Domain

In one aspect, provided is the fusion protein as defined herein, wherein the transmembrane domain may be obtained from a naturally occurring protein or a synthetic, non-naturally occurring protein segment, e.g., a hydrophobic protein segment that is thermodynamically stable in a cell membrane. In one aspect, the protein segment is at least approximately 20 amino acids, e.g., at least 18, 19, 20, 21, 22, 23, 24, 25, 26, 27, 28, 29, 30, or more amino acids.

In one aspect, the transmembrane domain provided herein is derived from a Type I single-pass membrane protein. In one aspect, the transmembrane domains from multi-pass membrane proteins may also be compatible for use in the fusion proteins described herein. Multi-pass membrane proteins may comprise a complex (at least 2, 3, 4, 5, 6, 7 or more) alpha helices or a beta sheet structure. In one aspect, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In one aspect, the transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In one aspect, one or more cysteine residues are present in the transmembrane region of the transmembrane domain. In one aspect, one or more cysteine residues are present in the cytoplasmic region of the transmembrane domain. In one aspect, the cytoplasmic region of the transmembrane domain comprises positively charged amino acids. In one aspect, the cytoplasmic region of the transmembrane domain comprises the amino acids arginine, serine, and lysine.

In one aspect, the transmembrane region of the transmembrane domain comprises hydrophobic amino acid residues. In one aspect, the transmembrane domain of the CAR provided herein comprises an artificial hydrophobic sequence. For example, a triplet of phenylalanine, tryptophan and valine may be present at the C terminus of the transmembrane domain. In one aspect, the transmembrane region comprises mostly hydrophobic amino acid residues, such as alanine, leucine, isoleucine, methionine, phenylalanine, tryptophan, or valine. In one aspect, the transmembrane region is hydrophobic. In some embodiments, the transmembrane region comprises a poly-leucine-alanine sequence.

In one aspect, the transmembrane domain comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), GITR, CD40, BAFFR, HVEM (LIGHTR), SLAMF7, NKp80 (KLRFI), CD160, GPC3, IL-2R beta, IL-2R gamma, IL-7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In one aspect, the transmembrane domain is derived from or comprises the transmembrane domain of CD8α, CD4, CD28, CD137, CD80, CD86, CD152 and PD1. In some specific embodiments, the transmembrane domain comprises the amino acid sequence having at least 88%, 92%, 96%, sequence identity to SEQ ID NO: 32. In a particular embodiment, the transmembrane domain comprises or consists of the amino acid sequence of SEQ ID NO: 32.

### 2.4 Intracellular Signaling Domain

In one aspect, provided is the fusion protein as defined herein, wherein the intracellular signaling domain is generally responsible for activation of at least one of the normal effector functions. In one aspect, the intracellular signaling domains include one or two distinct classes of cytoplasmic signaling sequences: those that initiate antigen-dependent primary activation through the TCR (primary intracellular signaling domains) and/or those that act in an antigen-independent manner to provide a secondary or co-stimulatory signal (secondary cytoplasmic domain, e.g., a co-stimulatory domain).

In one aspect, a primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs). Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FceRI, DAP10, DAP12, and CD66d. In one aspect, a CAR comprises an intracellular signaling domain, e.g., a primary signaling domain of CD3-zeta. In one aspect, a primary signaling domain comprises a modified ITAM domain, e.g., a mutated ITAM domain which has altered (e.g., increased or decreased) activity as compared to the native ITAM domain. In one aspect, a primary signaling domain comprises a modified ITAM-containing primary intracellular signaling domain, e.g., an optimized and/or truncated ITAM-containing primary intracellular signaling domain. In one aspect, the signaling domain of CD3-zeta is a mutant CD3zeta or a wild type human CD3zeta. In one aspect, a primary signaling domain comprises one, two, three, four or more ITAM motifs. In one aspect, the primary intracellular signaling domain is a functional mutant of the cytoplasmic signaling domain of CD33 containing one or more mutations, such as Q65K.

In one aspect, the intracellular signaling sequences within the cytoplasmic portion may be linked to each other in a random or specified order. In one aspect, the co-stimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, signaling lymphocytic activation molecules (SEAM proteins), activating NK cell receptors, BTFA, a Toll ligand receptor, OX40, CD2, CD7, CD27, CD28, CD30, CD40, CDS, ICAM-1, FFA-1 (CD11a/CD18), 4-1BB (CD137), B7-H3, CDS, ICAM-1, ICOS (CD278), GITR, BAFFR, FIGHT, HVEM, KIRDS2, SFAMF7, NKp80 (KFRF1), NKp44, NKp30, NKp46, CD19, CD4, CD8alpha, CD8beta, IF2Rbeta, IF2Rgamma, IF7R alpha, ITGA4, VFAI, CD49a, ITGA4, IA4, CD49D, ITGA6, VFA-6, CD49f, ITGAD, CD11d, ITGAE, CD103, ITGAF, FFA-1, ITGAM, CD11b, ITGAX, CD11c, ITGB1, CD29, ITGB2, CD18, FFA-1, ITGB7, NKG2D, NKG2C, TNFR2, TRANCE/RANKF, DNAM1 (CD226), SFAMF4 (CD244, 2B4), CD84, CD96 (Tactile), CEACAM1, CRTAM, Fy9 (CD229), CD160 (BY55), PSGF1, CD100 (SEMA4D), CD69, SFAMF6 (NTB-A, Fyl08), SEAM (SFAMF1, CD150, IPO-3), BFAME (SFAMF8), SEFPFG (CD162), FTBR, FAT, GADS, SFP-76, PAG/Cbp, CD19a, and a ligand that specifically binds with CD83, and the like.

In one aspect, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

In one aspect, the one or more co-stimulatory signaling domains are selected from the group consisting of the co-stimulatory signaling domains of CD27, CD28, CD137, OX40, CD30, CD40, CD3, lymphocyte function-associated antigen-1 (LFA-1), CD2, CD7, NKG2C, B7-H3 and ligands that specially bind to CD83. In one aspect, the intracellular signaling domain in the CAR of the present disclosure comprises a co-stimulatory signaling domain derived from CD137 (4-1BB). In one aspect, the intracellular signaling domain comprises a cytoplasmic signaling domain of CD32 and a co-stimulatory signaling domain of CD28 or CD137. In a particular embodiment, the intracellular signaling domain comprises a cytoplasmic signaling domain of CD32 and a co-stimulatory signaling domain of CD137.

### 2.5 CAR

In one aspect, provided is the fusion protein as defined herein, wherein the fusion protein is a CAR that comprises, from its N to C terminus in order: the extracellular antigen-binding domain, the spacer, the transmembrane domain and the intracellular signaling domain. In one aspect, the extracellular antigen-binding domain is an scFv that comprises, from its N to C terminus in order: a VL, the linker and a VH. In one aspect, the extracellular antigen-binding domain is an scFv that comprises or consists of the amino acid sequence of SEQ ID NO: 21, SEQ ID NO: 22 or SEQ ID NO: 23. In one aspect, the spacer consists of the amino acid sequence of SEQ ID NO: 28. In one aspect, the transmembrane domain comprises or consists of the amino acid sequence of SEQ ID NO: 32. In one aspect, the intracellular signaling domain comprises, from its N to C terminus in order: the co-stimulatory signaling domain and the primary intracellular signaling domain. In one aspect, the intracellular signaling domain comprises, from its N to C terminus in order: the signaling domain of CD28 or CD137 and the cytoplasmic signaling domain of CD35. In a particular embodiment, the CAR comprises or consists of the amino acid sequence of SEQ ID NO: 35 or SEQ ID NO: 36.

### III. Antibodies

The present disclosure also provides a novel antibody with advantageous properties such as producibility, stability, binding affinity, biological activity, targeting efficiency, reduced toxicity, an extended dosage range that can be given to a patient and thereby a possibly enhanced efficacy.

In one aspect, the antibody is an intact antibody or antibody fragment, such as Fv, Fab, Fab′, Fab′-SH, F(ab′)₂, diabodies, linear antibodies, single-chain antibody molecules (e.g. scFv and scFab), single-domain antibodies, or multispecific antibodies formed from antibody fragments.

In one aspect, the antibody is capable of binding to CD19. In one aspect, the antibody is capable of binding to the same epitope on CD19 as any anti-CD19 antibody in the art (e.g. FMC63). In one aspect, the antibody is capable of binding to the same epitope on CD19 as a reference antibody that comprises the amino acid sequence of SEQ ID NO: 21. In one aspect, the antibody comprises a VH and a VL, the VH comprises a HCDR1, a HCDR2 and a HCDR3 comprising the amino acid sequences of SEQ ID NO: 5, SEQ ID NO: 6 and SEQ ID NO: 7, respectively; the VL comprises a LCDR1, a LCDR2 and a LCDR3 comprising the amino acid sequences of SEQ ID NO: 8, SEQ ID NO: 9 and SEQ ID NO: 10, respectively.

In one aspect, the antibody comprises a VH and a VL, wherein the VH comprises the amino acid sequence of SEQ ID NO: 1; the VL comprises the amino acid sequence of SEQ ID NO: 2. In one aspect, the VH comprises the amino acid sequence having at least 90%, 91%, 92%, 93%, 94%, 95%, 96%, 97% or 98% sequence identity to SEQ ID NO: 1. In one aspect, the VL comprises the amino acid sequence having at least 90%, 91%, 92%, 93%, 94%, 95%, 96%, 97% or 98% sequence identity to SEQ ID NO: 2. In one aspect, the VL comprises the amino acid sequence having at least 90%, 91%, 92%, 93%, 94%, 95%, 96%, 97% or 98% sequence identity to SEQ ID NO: 2, wherein the amino acids of the VL at positions 39 and 80 are R and P, respectively. In one aspect, the VL comprises the amino acid sequence having at least 90%, 91%, 92%, 93%, 94%, 95% or 96% sequence identity to SEQ ID NO: 2, wherein the amino acids of the VL at positions 39, 80, 100 and 103 are R, P, S and R, respectively. In a particular embodiment, the VH comprises or consists of the amino acid sequence of SEQ ID NO: 1; the VL comprises or consists of the amino acid sequence of SEQ ID NO: 3 or SEQ ID NO: 4.

In one aspect, the VH and the VL are connected via a flexible polypeptide linker. In one aspect, the VH is fused by its N-terminus via a flexible polypeptide linker to the C-terminus of the VL (VL-VH). In one aspect, the VL is fused by its N-terminus via a flexible polypeptide linker to the C-terminus of the VH (VH-VL). In one aspect, the flexible polypeptide linker is (G₄S)ₙ, (SG₄)ₙ or G₄(SG₄)ₙ, wherein “n” is 1, 2, 3, 4, 5, 6, 7, 8, in particular 3. In one aspect, the flexible polypeptide linker comprises the amino acid sequence of SEQ ID NO: 19 or SEQ ID NO: 20. In one aspect, the antibody comprises the amino acid sequence having at least 90%, 91%, 92%, 93%, 94%, 95%, 96%, 97% or 98% sequence identity to SEQ ID NO: 21, SEQ ID NO: 22 or SEQ ID NO: 23. In one aspect, the antibody comprises the amino acid sequence of SEQ ID NO: 21, SEQ ID NO: 22 or SEQ ID NO: 23. CDRs and variable regions of exemplary antibodies are shown in column 2-9 of Table 3.

In one aspect, the present disclose also provides a complex comprising an antibody herein and a heterologous molecule or moiety. In one aspect, the heterologous molecule or moiety is covalently bound to the antibody. In one aspect, the complex is a fusion protein, e.g. a CAR. In one aspect, the complex comprises, from its N to C terminus in order: an extracellular antigen binding domain comprising the antibody, a transmembrane domain and an intracellular signaling domain, wherein the extracellular antigen binding domain comprises the antibody. The extracellular antigen binding domain, the transmembrane domain and intracellular signaling domain are recited in section II.

### IV. Engineered Cells and Preparation Thereof

### 4.1 Cells

In one aspect, the present disclosure provides engineered cells comprising aforementioned fusion proteins, e.g., engineered immune cells. The source of the engineered immune cells of the present disclosure may be a patient to be treated (i.e., autologous cells) or from a donor who is not the patient to be treated (e.g., allogeneic cells).

In one aspect, the cell is an immunoresponsive cell. In one aspect, the cell is a cell of the lymphoid lineage. In one aspect, the engineered immune cells are engineered T cells. In one aspect, the T cells are derived from a mammalian subject. In one aspect, the T cells are derived from a primate subject, such as a human subject. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, α/β T cells, and δ/γ T cells. Non-limiting examples of commercially available T-cell lines include lines BCL2 (AAA) Jurkat (ATCC″ CRL-2902™), BCL2 (S70A) Jurkat (ATCCR CRL-2900™), BCL2 (S87A) Jurkat (ATCC″ CRL-2901™), BCL2 Jurkat (ATCCB CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ [G11] (ATCC CRL-8294), HuT102 (ATCC TIB-162). Non-limiting exemplary sources for such commercially available cell lines include the American Type Culture Collection (ATCC) (Manassas, VA), and the German Collection of Microorganisms and Cell Cultures.

In certain aspects, the cells are cytotoxic T cells (also known as TC, Cytotoxic T Lymphocyte, CTL, T Killer cell, a lytic T cell, CD8+ T cells or killer T cell). In certain aspects, the T cell is a CD4+ T cell. In certain aspects, the T cell can be a CD4+ T cell or a CD8+ T cell. In certain aspects, the cell is a tumor-specific T cell.

In one aspect, the cells are natural killer (NK) cells, Natural Killer T (NKT) cells, cytokine-induced killer (CIK) cells, tumor-infiltrating lymphocytes (TILs), lymphokine-activated killer (LAK) cells, or the like. NK cells may either be isolated or obtained from a commercially available source. Non-limiting examples of commercial NK cell lines include lines NK-92 (ATCCR CRL-2407™), NK-92 MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection (ATCC) (Manassas, VA) and the German Collection of Microorganisms and Cell Cultures.

In one aspect, the cells are B cells, monocytes or granulocytes, e.g., myeloid cells, macrophages, neutrophils, dendritic cells, mast cells, eosinophils, and/or basophils.

In certain aspects, the cell is a cell of the myeloid lineage. Non-limiting examples of cells of the myeloid lineage include monocytes, macrophages, basophils, neutrophils, eosinophils, mast cell, erythrocytes, megakaryocytes, thrombocytes, and stem cells from which myeloid cells may be differentiated. In certain embodiments, the stem cell is a pluripotent stem cell (e.g., embryonic stem cell or induced pluripotent stem cell).

### 4.2. Methods for Genetic Engineering

In one aspect, the engineered cells are prepared by various methods of the transfer of polynucleotides encoding fusion proteins, e.g., antigen receptors, e.g., CARs. Physical methods include calcium phosphate precipitation, lipofection, particle bombardment, microinjection, electroporation, and the like. Biological methods include the use of DNA and RNA vectors, e.g., a viral vector, e.g., a lentiviral vector. Chemical methods include colloidal dispersion systems, such as macromolecule complexes, nanocapsules, microspheres, beads, and lipid-based systems including oil-in-water emulsions, micelles, mixed micelles, and liposomes. Exemplary methods are shown in Table 4. In some aspects, those well-known methods include transduction via viral e.g., retroviral or lentiviral, transposons, and electroporation.

TABLE 4
Delivery methods for the genome editing systems

| | | Duration of | Genome | Type of molecule |
|---|---|---|---|---|
| Delivery Mode | | Expression | integration | delivered |
| Physical (e.g. electroporation) | | Transient | No | Nucleic acids and |
| | | | | Proteins |
| Viral vector | retrovirus | Stable | Yes | RNA |
| | lentivirus | Stable | Yes | RNA |
| | adenovirus | Transient | No | DNA |
| | AAV | Stable | No | DNA |
| | Vaccinia Virus | Transient | No | DNA |
| | Herpes Simplex Virus | Stable | No | DNA |
| Non-viral | Cationic liposomes | Transient | | Nucleic acids and |
| | | | | Proteins |
| | Polymeric | Transient | | Nucleic acids and |
| | nanoparticles | | | Proteins |
| | Transposon | Stable | Yes | DNA |
| Biological non-viral | Attenuated bacteria | Transient | No | Nucleic acids |
| delivery vehicles | | | | |

In one aspect, recombinant polynucleotides are transferred into cells using recombinant infectious virus particles, e.g., adenoviral vector, AAV vector, lentiviral vector, retroviral vector, such as gamma-retroviral vectors. In one aspect, the retroviral vector or lentiviral vector has a long terminal repeat sequence (LTR). In one aspect, the vectors are self-inactivating (SIN). In one aspect, the vectors are conditionally replicating (mobilizable) vectors. In one aspect, the lentiviral vectors are derived from human, feline or simian lentiviruses. In one aspect, the retroviral vectors are derived from murine retroviruses. In one aspect, the lentiviruses or retroviruses include those derived from any avian or mammalian cell source. In one aspect, the lentiviruses or retroviruses are amphotropic, meaning that they are capable of infecting host cells of several species, including humans. In one aspect, the gene to be expressed replaces the retroviral gag, pol and/or env sequences.

In one aspect, the vector comprising the polynucleotides encoding the fusion protein e.g., CAR, can contain a promoter and/or enhancer or regulatory elements to regulate expression of the encoded recombinant receptor. In one aspect, the promoter and/or enhancer or regulatory elements can be condition-dependent promoters, enhancers, and/or regulatory elements. In one aspect, the polynucleotides encoding the fusion protein can be operatively linked to a constitutive promoter. In one aspect, the promoter is selected from the group consisting of Cytomegalovirus (CMV) promoter, elongation factors-1 alpha (EF1α) promoter, ubiquitin C (UbiC) promoter, phosphor-glycerokinase (PGK) promoter, simian virus 40 (SV40) early promoter and chicken β-Actin promoter coupled with CMV early enhancer (CAGG).

In one aspect, the polynucleotides are operably linked to an inducible promoter. The inducible promoter can be induced by one or more conditions, such as a physical condition, microenvironment of the engineered immune effector cell, or the physiological state of the engineered immune effector cell, an inducer (i.e., an inducing agent), or a combination thereof. In one aspect, the inducing condition does not induce the expression of endogenous genes in the engineered mammalian cell, and/or in the subject that receives the pharmaceutical composition. In one aspect, the inducing condition is selected from the group consisting of: inducer, irradiation (such as ionizing radiation, light), temperature (such as heat), redox state, tumor environment, and the activation state of the engineered mammalian cell.

In one aspect, the polynucleotides are operatively linked to a Woodchuck Hepatitis Virus (WHP) Posttranscriptional Regulatory Element (WPRE), located downstream of the polynucleotides.

In one aspect, the vector can contain a single promoter that drives the expression of one or more nucleic acid molecules. In one aspect, such nucleic acid molecules can be multi-cistronic. For example, in one aspect, transcription units can be engineered as a bi-cistronic unit containing an IRES (internal ribosome entry site), which allows co-expression of gene products (e.g., encoding a first and second CAR) by a message from a single promoter. In one aspect, a single promoter may direct expression of an RNA that contains, in a single open reading frame (ORF), two or three genes (e.g. encoding a first and second CAR) separated from one another by sequences encoding a self-cleavage peptide or a protease recognition site. In one aspect, the self-cleavage peptide is selected from the group consisting of foot-and-mouth disease virus (F2A), equine rhinitis A virus (E2A), Thosea asigna virus (T2A) and porcine teschovirus-1 (P2A).

In one aspect, polynucleotides are transferred into T cells via electroporation. In one aspect, polynucleotides are transferred into T cells via transposition. In one aspect, polynucleotides are delivered by means of transposons including a Sleeping Beauty transposon system (SB) and/or a piggyBac (PB) transposon system.

Polynucleotides encoding fusion proteins may be associated with additional coding regions which encode secretory or signal peptides, which direct the secretion of the fusion protein. For example, if secretion of the fusion protein is desired, DNA encoding a signal sequence may be placed upstream of the fusion protein. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In one aspect, the signal peptide comprises a sequence of a human CD2, CD38, CD38, CD3Y, CD35, CD4, CD8α, CD19, CD28, CD37, CD45, 4-1BB, GM-CSFR, IL-2, CD33, Human IgKVIII, Human IgG2 H, Chymotrypsinogen, trypsinogen-2, HSA, Insulin or tPA signal peptide. Exemplified signal peptides comprise or consist of the amino acid sequence of SEQ ID NO: 38 or SEQ ID NO: 39.

In one aspect, the polynucleotides encoding the fusion protein contain a nucleic acid sequence encoding one or more marker(s). In one aspect, the one or more marker(s) is a transduction marker, surrogate marker and/or a selection marker. In one aspect, the polynucleotides encoding the fusion protein contain a nucleic acid sequence encoding one or more additional fusion proteins that enhance and/or dampen responses of the cells upon adoptive transfer and encounter with ligand.

In one aspect, the polynucleotides can also encode one or more surrogate marker(s). In one aspect, the surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. In one aspect, the truncated cell surface polypeptides including truncated forms of growth factors or other receptors such as a truncated human epidermal growth factor receptor 2 (tHER2), a truncated epidermal growth factor receptor (EGFRt), a prostate-specific membrane antigen (PSMA) or modified form thereof. EGFRt can be used to identify or select cells that have been engineered with the EGFRt fusion protein and an encoded exogenous protein, and/or to eliminate or separate cells expressing the encoded exogenous protein.

In one aspect, the marker is a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In one aspect, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In one aspect, the marker is a selection marker. In one aspect, the selection marker is a polypeptide that confers resistance to exogenous agents or drugs. In one aspect, the selection marker is an antibiotic resistance gene. In one aspect, the selection marker is an antibiotic resistance gene confers antibiotic resistance to a mammalian cell. In one aspect, the selection marker is selected from the group consisting of a Puromycin resistance gene, a Hygromycin resistance gene, a Blasticidin resistance gene, a Neomycin resistance gene, a Geneticin resistance gene or a Zeocin resistance gene or a modified form thereof.

Alternatively, various assays can be used to confirm the presence of the recombinant DNA sequence in the engineered cell, such as Southern and Northern blotting, RT-PCR and PCR. In one aspect, the fusion protein can be detected by the ability to recognize target cells, or the release of cytokines (e.g., interferon-Y, granulocyte/monocyte colony stimulating factor (GM-CSF), tumor necrosis factor α (TNF-α) or interleukin 2 (IL-2)). In addition, the function of fusion protein can be evaluated by measurement of cellular cytotoxicity.

### 4.3 Preparation of Engineered Cells

In one aspect, the present disclosure provides a manufacture process of the engineered cells. In one aspect, any known method for preparation may be used. In a particular aspect, the method includes transducing a population of isolated cells with the polynucleotide encoding the fusion protein and selecting a subpopulation of said isolated cells that have been successfully transduced with the polynucleotide thereby producing genetically modified cells, as described above.

In one aspect, the method includes acquisition, isolation, transduction, expansion steps. In a particular aspect, the method includes the following steps: (i) acquisition of an immune cell population (e.g. blood cells) (ii) isolation of a particular cell population (e.g. T cells and/or NK cells) (iii) transducing a population of isolated cells with the polynucleotide encoding the fusion protein; and (iv) expanding a subpopulation of said isolated cells that have been successfully transduced with said nucleic acid sequence of step (iii) thereby producing genetically modified cells. These different steps are more particularly described below.

### 4.3.1 Cell Acquisition

In one aspect, the subject from which the cell is obtained for introduction of the fusion protein (e.g., CAR) is one having the disease or condition or in need of a cell therapy or to which cell therapy will be administered. In one aspect, the cells can be derived from a healthy donor.

In one aspect, the cells may be obtained from a sample, such as a biological sample. In one aspect, the samples are selected from whole blood, peripheral blood mononuclear cells (PBMCs), leukocytes, bone marrow, thymus, tissue biopsy, tumor tissue, leukemia tissue, lymphoma tissue, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In one aspect, the cells are primary cells. In one aspect, cells from the circulating blood of a subject are obtained, e.g., by apheresis or leukapheresis. The resulting samples comprise lymphocytes, including T cells, monocytes, granulocytes, B cells, other nucleated white blood cells, red blood cells, and/or platelets, and in some aspects contain cells other than red blood cells and platelets.

### 4.3.2 Cell Isolation

Various methods are readily available for isolating immune cells from a sample, for example using Life Technologies Dynabeads® system; STEMcell Technologies EasySep™, RoboSep™, RosetteSep™, SepMate™; Miltenyi Biotec MACS™ cell separation kits, cell surface marker expression and other commercially available cell separation and isolation kits (e.g., ISOCELL from Pierce, Rockford, IL). Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells.

In one aspect, isolation of the cells includes one or more non-affinity-based cell separation steps. In one aspect, cells are washed, centrifuged, and/or incubated in the presence of one or more reagents, for example, to remove unwanted components, enrich for desired components, lyse or remove cells sensitive to particular reagents. In one aspect, cells are separated based on one or more properties, such as density, adherent properties, size, sensitivity and/or resistance to particular components.

In one aspect, the blood cells collected from the subject are washed, e.g., to remove the plasma fraction and to place the cells in an appropriate buffer or media for subsequent processing steps. In one aspect, the cells are washed with phosphate buffered saline (PBS). In one aspect, the wash solution lacks calcium and may lack magnesium or may lack many if not all divalent cations. Initial activation steps in the absence of calcium may lead to magnified activation. In one aspect, a washing step is accomplished by a semi-automated “flow-through” centrifuge (for example, the Cobe 2991 cell processor, Baxter) according to the manufacturer's instructions. In one aspect, a washing step is accomplished by tangential flow filtration (TFF) according to the manufacturer's instructions. In one aspect, the cells are resuspended in a variety of biocompatible buffers after washing, such as, for example, Ca²⁺/Mg²⁺ free PBS. In certain embodiments, components of a blood cell sample are removed and the cells directly resuspended in culture media. In one aspect, the isolation includes density-based cell separation methods, such as the preparation of white blood cells from peripheral blood by lysing the red blood cells and centrifugation through a Percoll or Ficoll gradient.

In one aspect, the isolation methods include the separation of different cell types based on the expression or presence in the cell of one or more specific molecules, such as surface markers, e.g., surface proteins, intracellular markers, or nucleic acid. In one aspect, the separation is affinity- or immunoaffinity-based separation. The separation can be based on positive selection, in which the cells having bound the reagents are retained for further use, and/or negative selection, in which the cells having not bound to the antibody or binding partner are retained. The separation needs not result in 100% enrichment or removal of a particular cell population or cells expressing a particular marker.

In one aspect, one separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. In one aspect, multiple rounds of separation steps are carried out, where the positively or negatively selected fraction from one step is subjected to another separation step, such as a subsequent positive or negative selection.

In some aspects, specific subpopulations of T cells, such as cells positive or expressing high levels of one or more surface markers, e.g., CD3+, CD28+, CD62L+, CCR7+, CD27+, CD127+, CD4+, CD8+, CD45RA+, and/or CD45RO+ T cells, are isolated by positive or negative selection techniques. In one aspect, T cells are isolated by incubation with anti-CD3/anti-CD28 conjugated particles or beads (e.g., DYNABEADS® M-450 CD3/CD28 T Cell Expander, MACSiBeads™, etc.). In one aspect, the time period of positive selection is about 30 minutes. In a further embodiment, the time period is at least 1, 2, 3, 4, 5, or 6 hours. In one aspect, the time period is 10 to 24 hours. In one aspect, the incubation time period is 24 hours. For isolation of a desired population of cells by positive or negative selection, the concentration of cells and particles can be varied. In certain embodiments, it may be desirable to significantly decrease the volume in which beads and cells are mixed together (i.e., increase the concentration of cells), to ensure maximum contact of cells and beads. In one aspect, greater than 100 million cells/mL is used.

In some aspects, T cells are separated from a sample by negative selection of markers expressed on non-T cells, such as B cells, monocytes, or other white blood cells, such as CD14. In some aspects, a CD4 or CD8 selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some aspects, CD8+ cells are further enriched for or depleted of naive, central memory, effector memory, and/or central memory stem cells, such as by positive or negative selection based on surface antigens associated with the respective subpopulation. In some aspects, enrichment for central memory T (TCM) cells is carried out to increase efficacy, such as to improve long-term survival, expansion, and/or engraftment following administration. In some aspects, combining TCM-enriched CD8+ T cells and CD4+ T cells further enhances efficacy.

In some aspects, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some aspects, the enrichment for central memory T (TCM) cells is based on positive or high surface expression of CD45RO, CD62L, CCR7, CD28, CD3, and/or CD127. In some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

In one aspect, the enrichment for NK cells is based on positive or high surface expression of CD56 and CD16 and on the negative expression of CD3 and/or optionally on the presence of NKp46 or NKp30 receptors.

In one aspect, the sample or composition of cells to be separated is incubated with small, magnetizable or magnetically responsive material, such as magnetically responsive particles or microparticles, such as paramagnetic beads (e.g., Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select. In one aspect, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In one aspect, the magnetically responsive particles are left attached to the cells that are to be subsequently incubated, cultured and/or engineered; in some aspects, the particles are left attached to the cells for administration to a patient. In one aspect, the magnetically responsive particles are removed from the cells. Methods for removing magnetically responsive particles from cells are known and include, e.g., the use of competing non-labeled antibodies, magnetically responsive particles or antibodies conjugated to cleavable linkers, etc. In one aspect, the magnetically responsive particles are biodegradable.

In one aspect, the affinity-based selection is via magnetic-activated cell sorting (MACS®) (Miltenyi Biotec, Auburn, CA). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In one aspect, a cell population described herein is collected and enriched (or depleted) via flow cytometry, in which cells stained for multiple cell surface markers are carried in a fluidic stream. In one aspect, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system. In both cases, cells can be labeled with multiple markers, allowing for the isolation of well-defined T cell subsets at high purity.

In one aspect, the preparation methods include steps for freezing, e.g., cryopreserving, the cells, either before or after isolation, incubation, and/or engineering. In one aspect, the freeze and subsequent thaw step removes granulocytes and, to some extent, monocytes in the cell population. In one aspect, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank. In one aspect, cryopreserved cells are thawed and washed as described herein and allowed to rest for one hour at room temperature prior to activation.

### 4.3.3 Cell Expansion

In one aspect, the provided methods include cultivation, incubation, culture, and/or genetic engineering steps. In one aspect, the cells are incubated and/or cultured prior to or in connection with genetic engineering. The incubation steps can include culture, cultivation, stimulation, activation, and/or propagation. In one aspect, the cells are incubated in the presence of stimulating conditions or a stimulatory agent. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In one aspect, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or co-stimulatory receptor, e.g., anti-CD3, anti-CD28, for example, bound to solid support such as a bead (e.g., Dynabeads®), and/or one or more cytokines. In one aspect, a concentration of cells of 10, 15, 20, 25, 30, 35, 40, 45, 50, 75, 80, 85, 90, 95, 100 125 or 150 million cells/mL is used.

In one aspect, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and cells are cultured together for 2-3 days. Several cycles of stimulation may also be desired such that culture time of T cells can be 60 days or more. Conditions appropriate for T cell culture include an appropriate media that may contain factors necessary for proliferation and viability, including interleukin-2 (IL-2), insulin, IFN-γ, IL-4, IL-7, GM-CSF, IL-10, IL-12, IL-15, TGFß, and TNF-α or any other additives. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one aspect, NK cell populations can be expanded in vitro using interleukin-2 (IL-2) IL-15, IL-15/IL-15RA complex, IL-18 and IL-12. In one aspect, the NK cells are ex vivo expanded for at least about 5 days, for example, not less than about 10 days, not less than about 15 days, or not less than about 20 days before administration to the patient.

### V. Pharmaceutical Composition

Pharmaceutical compositions of the present disclosure may comprise engineered cells in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

In one aspect, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one aspect, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered one time or multiple times at these dosages. The cells can be administered by using infusion techniques that are commonly known in immunotherapy.

### VI. Method of Treatment

Pharmaceutical compositions of the present disclosure may be administered in a manner appropriate to the disease to be treated (or prevented). The diseases may comprise solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma, craniopharygioma, ependymoma, pinealoma, hemangioblastoma, acoustic neuroma, oligodendroglioma, menangioma, neuroblastoma, retinoblastoma and brain metastases); the non-solid tumors, such as leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

The quantity and frequency of administration will be determined by the condition of the patient, and the type and severity of the patient's disease, although appropriate dosages may be determined by clinical trials. The administration of the subject compositions may be carried out in any convenient manner, including by aerosol injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the compositions are directly injected into an organ of interest (e.g., an organ affected by a neoplasm). Alternatively, the compositions are provided indirectly to the organ of interest, for example, by administration into the circulatory system (e.g., the tumor vasculature). Expansion and differentiation agents can be provided prior to, during or after administration of the cells or compositions to increase production of T cells or NK cells in vitro or in vivo.

In one aspect, lymphodepletion is performed on a subject, e.g., prior to administering one or more cells described herein. In one aspect, the lymphodepletion comprises administering one or more of melphalan, cytoxan, cyclophosphamide, and fludarabine.

In one aspect, the engineered cells are administered as part of a combination treatment, such as simultaneously with or sequentially with, in any order, another therapeutic intervention, such as an antibody or engineered cell or receptor or agent, such as a cytotoxic or therapeutic agent. The cells or antibodies in some embodiments are co-administered with one or more additional therapeutic agents or in connection with another therapeutic intervention, either simultaneously or sequentially in any order. In one aspect, the cells are co-administered with another therapy sufficiently close in time such that the cell populations enhance the effect of one or more additional therapeutic agents, or vice versa. In one aspect, the cells or antibodies are administered prior to the one or more additional therapeutic agents. In one aspect, the cells or antibodies are administered after to the one or more additional therapeutic agents, such as anti-cancer agents. In the context of the present disclosure, it is contemplated that cell therapy could be used similarly in conjunction with chemotherapeutic, radiotherapeutic, or immunotherapeutic intervention, as well as pro-apoptotic or cell cycle regulating agents such as immune checkpoint inhibitor.

Alternatively, the present therapy may precede or follow the other agent treatment by intervals ranging from minutes to weeks. In embodiments where the other agent and the cell or antibody of the present disclosure are applied separately to the individual, one would generally ensure that a significant period of time did not expire between the times of each delivery, such that the agent and therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several week (1, 2, 3, 4, 5, 6, 7 or 8) lapse between the respective administrations. It is expected that the treatment cycles would be repeated if necessary. It also is contemplated that various standard therapies, as well as surgical intervention, may be applied in combination with the cell therapy.

### VII. Sequences

TABLE 5
Sequences of the present disclosure

| SEQ | | |
|---|---|---|
| ID No: | Name | Sequence |
| 1 | FMC63 VH | EVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYY |
| | | CAKHYYYGGSYAMDYWGQGTSVTVSS |
| 2 | FMC63 VL | DIQMTQTTSSLSASLGDRVTISCRASQDISKYLNWYQQKPDGTVKLLI |
| | | YHTSRLHSGVPSRFSGSGSGTDYSLTISNLEQEDIATYFCQQGNTLPY |
| | | TFGGGTKLEIT |
| 3 | VL-1 | DIQMTQTTSSLSASLGDRVTISCRASQDISKYLNWYQQRPDGTVKLLI |
| | | YHTSRLHSGVPSRFSGSGSGTDYSLTISNLEPEDIATYFCQQGNTLPYT |
| | | FGSGTRLEIT |
| 4 | VL-2 | DIQMTQTTSSLSASLGDRVTISCRASQDISKYLNWYQQRPDGTVKLLI |
| | | YHTSRLHSGVPSRFSGSGSGTDYSLTISNLEPEDIATYFCQQGNTLPYT |
| | | FGGGTKLEIT |
| 5 | FMC63 HCDR1 | DYGVS |
| 6 | FMC63 HCDR2 | VIWGSETTYYNSALKS |
| 7 | FMC63 HCDR3 | HYYYGGSYAMDY |
| 8 | FMC63 LCDR1 | RASQDISKYLN |
| 9 | FMC63 LCDR2 | HTSRLHS |
| 10 | FMC63 LCDR3 | QQGNTLPYT |
| 11 | SJ25C1 VH | EVKLQQSGAELVRPGSSVKISCKASGYAFSSYWMNWVKQRPGQGLE |
| | | WIGQIYPGDGDTNYNGKFKGQATLTADKSSSTAYMQLSGLTSEDSA |
| | | VYFCARKTISSVVDFYFDYWGQGTTVTVSS |
| 12 | SJ25C1 VL | DIELTQSPKFMSTSVGDRVSVTCKASQNVGTNVAWYQQKPGQSPKP |
| | | LIYSATYRNSGVPDRFTGSGSGTDFTLTITNVQSKDLADYFCQQYNR |
| | | YPYTSGGGTKLEI |
| 13 | SJ25C1 HCDR1 | SYWMN |
| 14 | SJ25C1 HCDR2 | QIYPGDGDTNYNGKFKG |
| 15 | SJ25C1 HCDR3 | KTISSVVDFYFDY |
| 16 | SJ25C1 LCDR1 | KASQNVGTNVA |
| 17 | SJ25C1 LCDR2 | SATYRNS |
| 18 | SJ25C1 LCDR3 | QQYNRYPYT |
| 19 | linker-1 | GSTSGSGKPGSGEGSTKG |
| 20 | linker-2 | GGGGSGGGGSGGGGS |
| 21 | FMC63 scFv | DIQMTQTTSSLSASLGDRVTISCRASQDISKYLNWYQQKPDGTVKLLI |
| | | YHTSRLHSGVPSRFSGSGSGTDYSLTISNLEQEDIATYFCQQGNTLPY |
| | | TFGGGTKLEITGSTSGSGKPGSGEGSTKGEVKLQESGPGLVAPSQSLS |
| | | VTCTVSGVSLPDYGVSWIRQPPRKGLEWLGVIWGSETTYYNSALKS |
| | | RLTIIKDNSKSQVFLKMNSLQTDDTAIYYCAKHYYYGGSYAMDYW |
| | | GQGTSVTVSS |
| 22 | scFv-1 | DIQMTQTTSSLSASLGDRVTISCRASQDISKYLNWYQQRPDGTVKLLI |
| | | YHTSRLHSGVPSRFSGSGSGTDYSLTISNLEPEDIATYFCQQGNTLPYT |
| | | FGSGTRLEITGSTSGSGKPGSGEGSTKGEVKLQESGPGLVAPSQSLSV |
| | | TCTVSGVSLPDYGVSWIRQPPRKGLEWLGVIWGSETTYYNSALKSRL |
| | | TIIKDNSKSQVFLKMNSLQTDDTAIYYCAKHYYYGGSYAMDYWGQ |
| | | GTSVTVSS |
| 23 | scFv-2 | DIQMTQTTSSLSASLGDRVTISCRASQDISKYLNWYQQRPDGTVKLLI |
| | | YHTSRLHSGVPSRFSGSGSGTDYSLTISNLEPEDIATYFCQQGNTLPYT |
| | | FGGGTKLEITGSTSGSGKPGSGEGSTKGEVKLQESGPGLVAPSQSLSV |
| | | TCTVSGVSLPDYGVSWIRQPPRKGLEWLGVIWGSETTYYNSALKSRL |
| | | TIIKDNSKSQVFLKMNSLQTDDTAIYYCAKHYYYGGSYAMDYWGQ |
| | | GTSVTVSS |
| 24 | scFv-3 | EVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYY |
| | | CAKHYYYGGSYAMDYWGQGTSVTVSSGSTSGSGKPGSGEGSTKGDI |
| | | QMTQTTSSLSASLGDRVTISCRASQDISKYLNWYQQKPDGTVKLLIY |
| | | HTSRLHSGVPSRFSGSGSGTDYSLTISNLEQEDIATYFCQQGNTLPYTF |
| | | GGGTKLEIT |
| 25 | SJ25C1 scFv | EVKLQQSGAELVRPGSSVKISCKASGYAFSSYWMNWVKQRPGQGLE |
| | | WIGQIYPGDGDTNYNGKFKGQATLTADKSSSTAYMQLSGLTSEDSA |
| | | VYFCARKTISSVVDFYFDYWGQGTTVTVSSGGGGSGGGGSGGGGSD |
| | | IELTQSPKFMSTSVGDRVSVTCKASQNVGTNVAWYQQKPGQSPKPLI |
| | | YSATYRNSGVPDRFTGSGSGTDFTLTITNVQSKDLADYFCQQYNRYP |
| | | YTSGGGTKLEI |
| 26 | S-1 | ESKYGPPSPPCP |
| 27 | S-2 | EPKSCDKTHTCP |
| 28 | S-3 | ESKSCDTPPPCPRCP |
| 29 | S-4 | ESKYGPPPCPRCP |
| 30 | S-W | ESKYGPPCPPCP |
| 31 | S-M | EXKSCDTPPPXPXCP |
| 32 | CD28 | MFWVLVVVGGVLACYSLLVTVAFIIFWV |
| | transmembrane | |
| | domain | |
| 33 | 4-1BB co- | KRGRKKLLYIFKQPFMRPVQTTQEEDGCSCRFPEEEEGGCEL |
| | stimulatory | |
| | domain | |
| 34 | CD3ζ | RVKFSRSADAPAYQQGQNQLYNELNLGRREEYDVLDKRRGRDPEM |
| | | GGKPRRKNPQEGLYNELQKDKMAEAYSEIGMKGERRRGKGHDGLY |
| | | QGLSTATKDTYDALHMQALPPR |
| 35 | CAR-3 | DIQMTQTTSSLSASLGDRVTISCRASQDISKYLNWYQQRPDGTVKLLI |
| | | YHTSRLHSGVPSRFSGSGSGTDYSLTISNLEPEDIATYFCQQGNTLPYT |
| | | FGSGTRLEITGSTSGSGKPGSGEGSTKGEVKLQESGPGLVAPSQSLSV |
| | | TCTVSGVSLPDYGVSWIRQPPRKGLEWLGVIWGSETTYYNSALKSRL |
| | | TIIKDNSKSQVFLKMNSLQTDDTAIYYCAKHYYYGGSYAMDYWGQ |
| | | GTSVTVSSESKSCDTPPPCPRCPMFWVLVVVGGVLACYSLLVTVAFII |
| | | FWVKRGRKKLLYIFKQPFMRPVQTTQEEDGCSCRFPEEEEGGCELRV |
| | | KFSRSADAPAYQQGQNQLYNELNLGRREEYDVLDKRRGRDPEMGG |
| | | KPRRKNPQEGLYNELQKDKMAEAYSEIGMKGERRRGKGHDGLYQG |
| | | LSTATKDTYDALHMQALPPR |
| 36 | CAR-7 | DIQMTQTTSSLSASLGDRVTISCRASQDISKYLNWYQQRPDGTVKLLI |
| | | YHTSRLHSGVPSRFSGSGSGTDYSLTISNLEPEDIATYFCQQGNTLPYT |
| | | FGGGTKLEITGSTSGSGKPGSGEGSTKGEVKLQESGPGLVAPSQSLSV |
| | | TCTVSGVSLPDYGVSWIRQPPRKGLEWLGVIWGSETTYYNSALKSRL |
| | | TIIKDNSKSQVFLKMNSLQTDDTAIYYCAKHYYYGGSYAMDYWGQ |
| | | GTSVTVSSESKSCDTPPPCPRCPMFWVLVVVGGVLACYSLLVTVAFII |
| | | FWVKRGRKKLLYIFKQPFMRPVQTTQEEDGCSCRFPEEEEGGCELRV |
| | | KFSRSADAPAYQQGQNQLYNELNLGRREEYDVLDKRRGRDPEMGG |
| | | KPRRKNPQEGLYNELQKDKMAEAYSEIGMKGERRRGKGHDGLYQG |
| | | LSTATKDTYDALHMQALPPR |
| 37 | CAR-11 | EVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYY |
| | | CAKHYYYGGSYAMDYWGQGTSVTVSSGSTSGSGKPGSGEGSTKGDI |
| | | QMTQTTSSLSASLGDRVTISCRASQDISKYLNWYQQKPDGTVKLLIY |
| | | HTSRLHSGVPSRFSGSGSGTDYSLTISNLEQEDIATYFCQQGNTLPYTF |
| | | GGGTKLEITESKSCDTPPPCPRCPMFWVLVVVGGVLACYSLLVTVAF |
| | | IIFWVKRGRKKLLYIFKQPFMRPVQTTQEEDGCSCRFPEEEEGGCELR |
| | | VKFSRSADAPAYQQGQNQLYNELNLGRREEYDVLDKRRGRDPEMG |
| | | GKPRRKNPQEGLYNELQKDKMAEAYSEIGMKGERRRGKGHDGLYQ |
| | | GLSTATKDTYDALHMQALPPR |
| 38 | signal peptide-1 | MLLLVTSLLLCELPHPAFLLIP |
| 39 | signal peptide-2 | METDTLLLWVLLLWVPGSTG |
| 40 | EGFRt | MLLLVTSLLLCELPHPAFLLIPRKVCNGIGIGEFKDSLSINATNIKHFK |
| | | NCTSISGDLHILPVAFRGDSFTHTPPLDPQELDILKTVKEITGFLLIQA |
| | | WPENRTDLHAFENLEIIRGRTKQHGQFSLAVVSLNITSLGLRSLKEISD |
| | | GDVIISGNKNLCYANTINWKKLFGTSGQKTKIISNRGENSCKATGQV |
| | | CHALCSPEGCWGPEPRDCVSCRNVSRGRECVDKCNLLEGEPREFVE |
| | | NSECIQCHPECLPQAMNITCTGRGPDNCIQCAHYIDGPHCVKTCPAG |
| | | VMGENNTLVWKYADAGHVCHLCHPNCTYGCTGPGLEGCPTNGPKI |
| | | PSIATGMVGALLLLLVVALGIGLFM |
| 41 | T2A | EGRGSLLTCGDVEENPGP |
| 42 | linker-3 | (G₄S)ₙ, wherein “n” is 1, 2, 3, 4, 5, 6, 7 or 8, in |
| | | particular 3. |
| 43 | linker-4 | (SG₄)ₙ, wherein “n” is 1, 2, 3, 4, 5, 6, 7 or 8, in |
| | | particular 3. |
| 44 | linker-5 | G₄(SG₄)ₙ, wherein “n” is 1, 2, 3, 4, 5, 6, 7 or 8, |
| | | in particular 3. |

### VIII. Example

The following examples are included for illustrative purposes only and are not intended to limit the scope of the invention.

### Recombinant DNA Techniques

Standard methods were used to manipulate DNA as described in Sambrook et al, Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al, (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments, where required, were either generated by PCR using appropriate templates or were synthesized from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow subcloning into the respective expression vectors. All constructs were designed with a 5′-end DNA sequence coding for a leader peptide which targets proteins for secretion in eukaryotic cells.

### Protein Expression

Proteins were expressed in host cells for several days as required. Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Protein Purification

Proteins were purified from filtered cell culture supernatants referring to standard protocols. In brief, antibodies were applied to a Protein A Sepharose column and washed with PBS. Elution of antibodies was achieved at low pH followed by immediate neutralization of the sample.

### Example 1: Generation and Assessment of Anti-CD19 Antibodies

Antibodies comprising FMC63 scFv, scFv-1, scFv-2 or scFv-3 (sequences shown in Table. 3) fused with human IgG1 Fc fragments were synthetized and examined for affinity to cognate ligand CD19 by surface plasmon resonance (SPR).

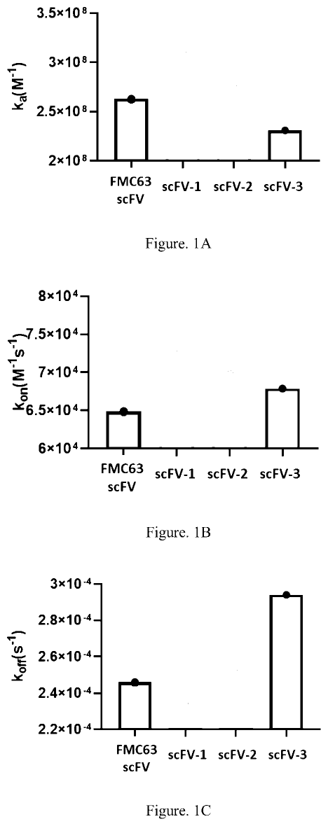

All SPR experiments were performed on a Biacore T200 (PCytiva) with HBS-EP as running buffer. The antibodies were captured by Protein A chip (LOT #10309880, Cytiva). Soluble CD19 protein (amino acids 20-291) was used as the analyte at concentrations ranging from 300 nM to 4.69 nM through the flow cells for 120 seconds followed by dissociation analysis for 300 seconds. Association rates (Kon), dissociation rates (Koff) and the constant Ka (Kon/Koff) were fitted to the results. As can be seen in FIG. 1, the antibodies comprising scFv-1 and scFv-2 had improved affinity compared to the other binders.

### Example 2: Generation and Assessment of CD19 CARs

CARs in Table 6 with signal peptides were formed through the fusion of a signal peptide (signal peptide-1 for CAR-W, signal peptide-2 for CAR-1 to CAR-13) to either FMC63 scFv, scFv-1, scFv-2 or scFv-3 followed by four different spacers and a transmembrane derived from human CD28 and an endodomain encoding the signaling motifs from 41BB and CD3λ. Each CAR was co-expressed with a truncated EGFR (EGFRt, the sequence is shown in SEQ ID NO: 40), use as a transduction marker. Both CAR and EGFRt were encoded from a single promoter construct separated by a self-cleaving T2A peptide.

TABLE 6
Structures of candidate CARs (from N- to C-terminus)

| | | | | | Primary |
|---|---|---|---|---|---|
| | Antigen- | | Trans- | Co- | intracellular |
| | binding | | membrane | stimulatory | signaling |
| No | domain | Spacer | domain | domain | domain |
| CAR-W | FMC63 | S-W | CD28 | 4-1BB co- | CD3ζ |
| | scFv | | trans- | stimulatory | intracellular |
| CAR-1 | scFv-1 | S-1 | membrane | domain | signaling |
| CAR-2 | scFv-1 | S-2 | domain | | domain |
| CAR-3 | scFv-1 | S-3 | | | |
| CAR-4 | scFv-1 | S-4 | | | |
| CAR-5 | scFv-2 | S-1 | | | |
| CAR-6 | scFv-2 | S-2 | | | |
| CAR-7 | scFv-2 | S-3 | | | |
| CAR-8 | scFv-2 | S-4 | | | |
| CAR-9 | scFv-3 | S-1 | | | |
| CAR-10 | scFv-3 | S-2 | | | |
| CAR-11 | scFv-3 | S-3 | | | |
| CAR-12 | scFv-3 | S-4 | | | |
| CAR-13 | scFv-3 | S-W | | | |

Primary human T cell populations expressing the various CARs were generated. Nucleic acid molecules encoding each fusion protein were individually cloned into a lentiviral vector. T cells, isolated from human PBMC samples obtained from healthy donors, were stimulated with CD3/CD28 beads (DYNABEAD®). One day after the stimulation, the cells were transduced with lentiviral vector, followed by expansion for 6 days. Cells were stained with EGFR-AF488 and CD19ₚᵣₒ-APC and examined by flow cytometry. CD19 binding capacity was used as a surrogate for CAR stability and affinity and examined by analyzing the mean florescent index (MFI) from the CD19 stain after gating on EGFRt positive cells.

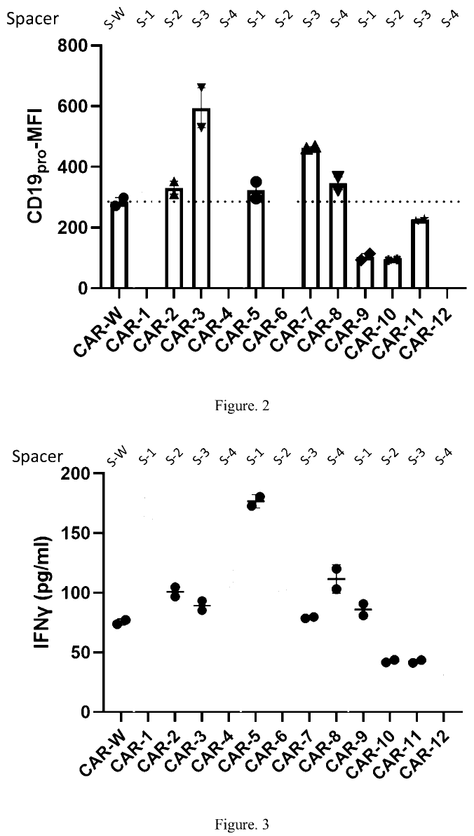

CARs comprising Spacer S-3 (CAR-3, CAR-7 and CAR-11) have an increased capacity to bind CD19 compared to other spacers carrying the same binder (FIG. 2). CAR-3 displayed the highest binding capacity.

### Example 3: Tonic Signaling of CD19 CAR Constructs

Certain CARs can exhibit antigen-independent activity or signaling, also known as tonic signaling. Excessive tonic signaling could lead to increased differentiation and T cell exhaustion. To assess tonic signaling of the transduced CAR T cells of Example 2, cells were left for 8 days in co-culture in the absence of exogenous stimulation. IFNγ concentration was then measured by Perkin Elmer AlphaLISA detection kit according to the manufacturer's instructions.

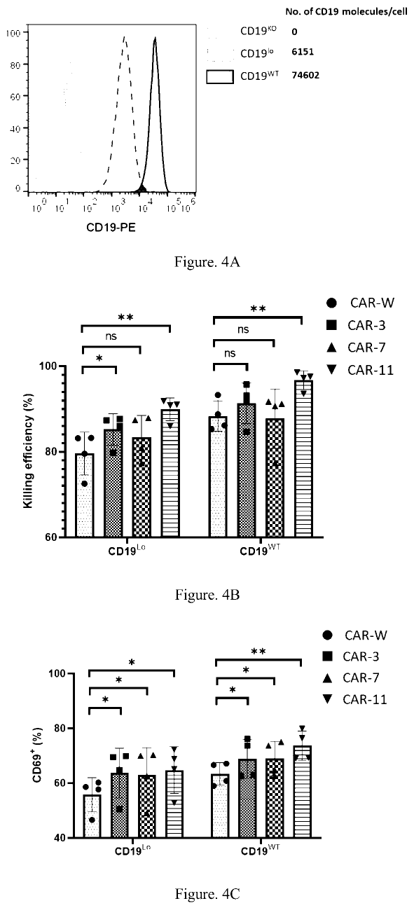

In FIG. 3, CARs comprising Spacer S-3 (CAR-3, CAR-7 and CAR-11) exhibited low levels of tonic signaling, as indicated by the low level of antigen independent IFNγ secretion.

### Example 4: Killing Efficiency and CD69 Expression of CD19 CAR Constructs

Primary human T-cells from 4 healthy donors were transduced with lentiviral vectors comprising the polynucleotides encoding the fusion protein CAR-W, CAR-3, CAR-7 or CAR-11 in Example 2. Transduction efficiency, as assessed by EGFR staining, was normalized to 50% for all samples through the addition of non-transduced cells. Cytotoxicity was then examined after these transduced cells were challenged with Raji target cells engineered to express approximately 6,704 copies or 74,602 copies of CD19 on their cell surface (FIG. 4A). The effector cells or the vector control cells expressing EGFRt only were co-cultured with the target cells at the E:T of 1:1. After 20 hours, the cells were isolated and stained with CD3, EGFR and CD20 and CD69.

The killing efficiency for each CAR T cell sample was calculated as a percentage of remaining target cells co-cultured with T cells transduced with the EGFRt only. As shown in FIG. 4B, the killing efficiencies of CAR-3 and CAR-11 is greater than those observed for CAR-W. All transduced CAR T cells showed comparable levels of CD69 expression (FIG. 4C).

### Example 5: Cytokine Release and T Cell Expansion Post Target Cell Stimulation

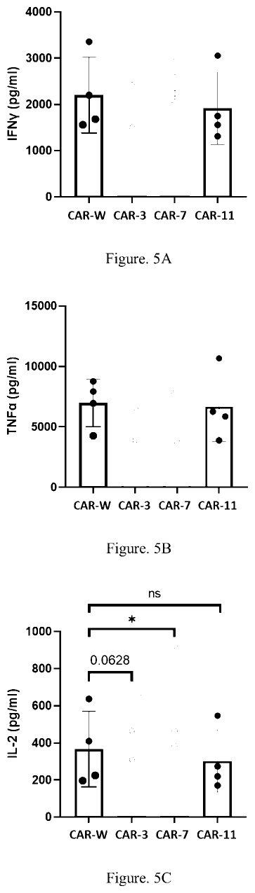

The effector cells of Example 4 were used to assess the cytokine release. The supernatants in example 4 were collected and analyzed for accumulated IFN-γ, TNF-α, and IL-2 cytokines. The results are depicted in FIG. 5A-5C. Cells transduced with CAR-3, CAR-7 and CAR-11 showed similar IFNγ and TNFα secretion after being challenged with tumor cells. However, CAR-3 and CAR-7 produced greater IL-2.

In an extended assay of example 4, the T cells were allowed to proliferate for 7 days after the initial stimulation with target cells and the number of CAR T cells was assessed. As shown in FIG. 5D, T cells transduced with CAR-3 and CAR-7 showed significantly higher numbers of T cells compared to the other CAR constructs.

### Example 6: Killing Efficiency of CD19 CARs at Various E:T Ratios

Primary human T-cells from 3 healthy donors were transduced with lentiviral vectors comprising the polynucleotides encoding the fusion protein CAR-W or CAR-13 in Example 1-2 (effector cells) or a lentiviral vector comprising the polynucleotide encoding EGFRt. Transduction efficiency, as assessed by EGFR staining, was normalized to 50% for all samples through the addition of non-transduced cells. Cytotoxicity was then examined after these effector cells were challenged with Raji-CD194^{Lo} target cells at various E:T ratios. The effector cells or the vector control cells expressing EGFRt only were co-cultured with Raji-CD19^{Lo} target cells at the E:T ratio of 1:1, 1:2, 1:8. At day 3 of co-culture assay, the cell samples were isolated and stained with CD3, EGFR and CD20.

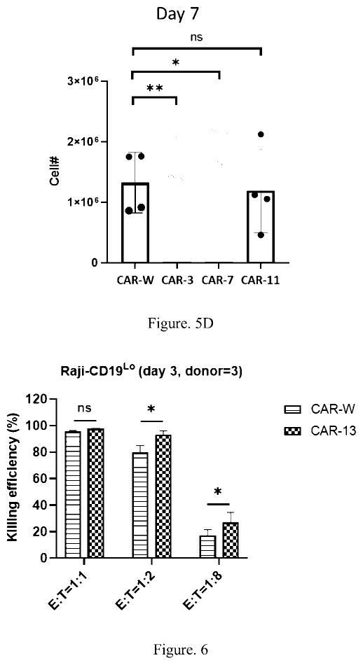

The killing efficiency for each CAR-T cell sample was calculated as a percentage of remaining target cells co-cultured with T cells transduced with the EGFRt only. As shown in FIG. 6, the killing efficiencies of both CAR-W and CAR-13 decrease with the increase of E:T ratios. However, CAR-13 shows greater elimination of Raji-CD19^{Lo} target cells than CAR-W on the ET ratio of 1:2 (p=0.0129, CAR-13 vs. CAR-W) and 1:8 (p=0.0367, CAR-13 vs. CAR-W). This data shows the enhanced anti-tumor activity of CAR-T cells through the S-3 spacer.

1. A fusion protein comprising
a) an extracellular antigen-binding domain;
b a spacer that consists of the amino acid sequence of SEQ ID NO: 28 or a functional equivalent thereof;
c) a transmembrane domain; and
d) an intracellular signaling domain.

2. The fusion protein of claim 1, wherein the spacer consists of the amino acid sequence of SEQ ID NO: 31 (EX₁KSCDTPPPX₂PX₃CP); wherein X₁ is S or P; X₂ is C or S; and X₃ is R or P;
preferably, the spacer consists of the amino acid sequence of SEQ ID NO: 31 (EX₁KSCDTPPPX₂PX₃CP); wherein X₁ is S; X₂ is C or S; and X₃ is R or P;
more preferably, the spacer consists of the amino acid sequence of SEQ ID NO: 28.

3. The fusion protein of claim 1 or 2, wherein the extracellular antigen-binding domain comprises an antibody comprising a heavy chain variable region (VH) and a light chain variable region (VL);
preferably, the antibody is capable of binding to the same epitope on CD19 as a reference antibody that comprises the amino acid sequence of SEQ ID NO: 21 or SEQ ID NO: 25;
more preferably, the VH comprises a HCDR1, a HCDR2 and a HCDR3 comprising the amino acid sequences of SEQ ID NO: 5, SEQ ID NO: 6 and SEQ ID NO: 7, respectively; the VL comprises a LCDR1, a LCDR2 and a LCDR3 comprising the amino acid sequences of SEQ ID NO: 8, SEQ ID NO: 9 and SEQ ID NO: 10, respectively; or
the VH comprises a HCDR1, a HCDR2 and a HCDR3 comprising the amino acid sequences of SEQ ID NO: 13, SEQ ID NO: 14 and SEQ ID NO: 15, respectively; the VL comprises a LCDR1, a LCDR2 and a LCDR3 comprising the amino acid sequences of SEQ ID NO: 16, SEQ ID NO: 17 and SEQ ID NO: 18, respectively.

4. The fusion protein of claim 3, wherein the VH comprises the amino acid sequence of SEQ ID NO: 1; the VL comprises the amino acid sequence of SEQ ID NO: 2, or the amino acid sequence having at least 90% sequence identity to SEQ ID NO: 2, wherein the amino acids of the VL at positions 39 and 80 are R and P, respectively;
preferably, the VL comprises the amino acid sequence having at least 95% identity to SEQ ID NO: 2, optionally wherein the amino acids of the VL at positions 100 and 103 are S and R, respectively;
more preferably, the VH comprises the amino acid sequence of SEQ ID NO: 1; the VL comprises the amino acid sequence of SEQ ID NO: 3 or SEQ ID NO: 4.

5. The fusion protein of claim 3 or 4, wherein the antibody is an scFv, the VH is fused by its N-terminus via a flexible polypeptide linker to the C-terminus of the VL;
preferably, the flexible polypeptide linker comprises the amino acid sequence of SEQ ID NO: 19 or SEQ ID NO: 20;
more preferably, the scFv comprises the amino acid sequence of SEQ ID NO: 21, SEQ ID NO: 22 or SEQ ID NO: 23.

6. The fusion protein of any one of claims 1 to 5, wherein the transmembrane domain comprises a transmembrane domain of CD8α, CD4, CD28, CD137, CD80, CD86, CD152 or PD1;
preferably, the transmembrane domain comprises a transmembrane domain of CD8α, CD4 or CD28;
more preferably, the transmembrane domain comprises the amino acid sequence of SEQ ID NO: 32.

7. The fusion protein of any one of claims 1 to 6, wherein the intracellular signaling domain comprises a co-stimulatory signaling domain;
preferably, the co-stimulatory signaling domain is selected from the group consisting of a signaling domain of CD27, CD28, CD137, OX40, CD30, CD40, CD3, HVEM, ICOS, Myd88, LFA-1, ICOS, CD2, CD7, NKG2C, B7-H3, Ligands of CD83 and a combination thereof;
more preferably, the co-stimulatory signaling domain is a signaling domain of CD28 or CD137.

8. The fusion protein of any one of claims 1 to 7, wherein the intracellular signaling domain comprises a primary intracellular signaling domain;
preferably, the primary intracellular signaling domain comprises a cytoplasmic signaling domain of CD3ζ;
more preferably, the intracellular signaling domain comprises a co-stimulatory signaling domain fused by its C-terminus to the N-terminus of the primary intracellular signaling domain, wherein the co-stimulatory signaling domain is a signaling domain of CD137; the primary intracellular signaling domain comprises a cytoplasmic signaling domain of CD3ζ.

9. The fusion protein of any one of claims 1 to 8, wherein the fusion protein is a CAR that comprises, from its N to C terminus in order: the extracellular antigen-binding domain, the spacer, the transmembrane domain and the intracellular signaling domain;
preferably, the extracellular antigen-binding domain comprises the amino acid sequence of SEQ ID NO: 21, SEQ ID NO: 22 or SEQ ID NO: 23, the spacer consists of the amino acid sequence of SEQ ID NO: 28;
more preferably, the CAR comprises the amino acid sequence of SEQ ID NO: 35 or SEQ ID NO: 36.

10. An antibody that binds to CD19 comprising a VH and a VL, wherein the VH comprises the amino acid sequence of SEQ ID NO: 1; the VL comprises an amino acid sequence having at least 90% identity to SEQ ID NO: 2, wherein the amino acids of the VL at positions 39 and 80 are R and P, respectively;
preferably, the VL comprises an amino acid sequence having at least 95% identity to SEQ ID NO: 2, optionally wherein the amino acids of the VL at positions 39 and 80 are R and P, respectively; optionally the amino acids of the VL at positions 100 and 103 are S and R, respectively;
more preferably, the VH comprises the amino acid sequence of SEQ ID NO: 1; the VL comprises the amino acid sequence of SEQ ID NO: 3 or SEQ ID NO: 4.

11. The antibody of claim 10, wherein the antibody is an scFv, the VH is fused by its N-terminus via a flexible polypeptide linker to the C-terminus of the VL;
preferably, the flexible polypeptide linker comprises the amino acid sequence of SEQ ID NO: 19 or SEQ ID NO: 20;
more preferably, the antibody comprises the amino acid sequence of SEQ ID NO: 22 or SEQ ID NO: 23.

12. A complex, comprising the antibody of claim 10 or 11 and a heterologous molecule or moiety;
preferably, the heterologous molecule or moiety is covalently bound to the antibody;
more preferably, the complex is a fusion protein comprising, from its N to C terminus in order: an extracellular antigen binding domain comprising the antibody, a transmembrane domain and an intracellular signaling domain.

13. An isolated nucleic acid encoding the fusion protein of any one of claims 1-9 or the antibody of claim 10 or 11.

14. A vector comprising the nucleic acid of claim 13, preferably, the vector is a viral vector, more preferable, the viral vector is a retroviral, lentiviral, adenoviral, or adeno-associated viral vector.

15. A cell comprising the fusion protein of any one of claims 1-9, the antibody of claim 10 or 11, the complex of claim 12, the isolated nucleic acid of claim 13, or the vector of claim 14, preferably, the cell is a lymphocyte, more preferable, the cell is a NK cell or a T cell.

16. A pharmaceutical formulation comprising the fusion protein of any one of claims 1-9, the antibody of claim 10 or 11, the complex of claim 12 or the cell of claim 15 and a pharmaceutically acceptable carrier.

17. A method of treating a cancer or an autoimmune disease in a subject, comprising administering the fusion protein of any one of claims 1-9, the antibody of claim 10 or 11, the complex of claim 12 or the cell of claim 15 to the subject,
preferably, the cancer is selected from the group consisting of leukemia, lymphoma, lung cancer, melanoma, breast cancer, prostate cancer, colon cancer, renal cell carcinoma, ovarian cancer, neuroblastoma and rhabdomyosarcoma, the autoimmune disease is selected from the group consisting of systemic lupus erythematosus, lupus nephritis, multiple sclerosis, rheumatoid arthritis, Sjogren's syndrome, idiopathic thrombocytopenia purpura, Type 1 diabetes, Pemphigus vulgaris, Neuromyelitis optica, ANCA vasculitis, and Myasthenia gravis;
more preferable, the cancer is leukemia or lymphoma; the autoimmune disease is systemic lupus erythematosus or lupus nephritis.


SEQUENCE LISTING (44 sequences)

SEQ ID NO: 1 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 3 - protein, 107 aa, synthetic construct
DIQMTQTTSSLSASLGDRVTISCRASQDISKYLNWYQQRPDGTVKLLIYHTSRLHSGVPSRFSGSGSGTDYSLTISNLEPEDIATYFCQQGNTLPYTFGSGTRLEIT

SEQ ID NO: 4 - protein, 107 aa, synthetic construct
DIQMTQTTSSLSASLGDRVTISCRASQDISKYLNWYQQRPDGTVKLLIYHTSRLHSGVPSRFSGSGSGTDYSLTISNLEPEDIATYFCQQGNTLPYTFGGGTKLEIT

SEQ ID NO: 5 - protein, 5 aa, synthetic construct
DYGVS

SEQ ID NO: 6 - protein, 16 aa, synthetic construct
VIWGSETTYYNSALKS

SEQ ID NO: 7 - protein, 12 aa, synthetic construct
HYYYGGSYAMDY

SEQ ID NO: 8 - protein, 11 aa, synthetic construct
RASQDISKYLN

SEQ ID NO: 9 - protein, 7 aa, synthetic construct
HTSRLHS

SEQ ID NO: 10 - protein, 9 aa, synthetic construct
QQGNTLPYT

SEQ ID NO: 11 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 12 - protein, 106 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 13 - protein, 5 aa, synthetic construct
SYWMN

SEQ ID NO: 14 - protein, 17 aa, synthetic construct
QIYPGDGDTNYNGKFKG

SEQ ID NO: 15 - protein, 13 aa, synthetic construct
KTISSVVDFYFDY

SEQ ID NO: 16 - protein, 11 aa, synthetic construct
KASQNVGTNVA

SEQ ID NO: 17 - protein, 7 aa, synthetic construct
SATYRNS

SEQ ID NO: 18 - protein, 9 aa, synthetic construct
QQYNRYPYT

SEQ ID NO: 19 - protein, 18 aa, synthetic construct
GSTSGSGKPGSGEGSTKG

SEQ ID NO: 20 - protein, 15 aa, synthetic construct
GGGGSGGGGSGGGGS

SEQ ID NO: 21 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - protein, 245 aa, synthetic construct
DIQMTQTTSSLSASLGDRVTISCRASQDISKYLNWYQQRPDGTVKLLIYHTSRLHSGVPSRFSGSGSGTDYSLTISNLEPEDIATYFCQQGNTLPYTFGSGTRLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - protein, 245 aa, synthetic construct
DIQMTQTTSSLSASLGDRVTISCRASQDISKYLNWYQQRPDGTVKLLIYHTSRLHSGVPSRFSGSGSGTDYSLTISNLEPEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 24 - protein, 245 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 25 - protein, 243 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 26 - protein, 12 aa, synthetic construct
ESKYGPPSPPCP

SEQ ID NO: 27 - protein, 12 aa, synthetic construct
EPKSCDKTHTCP

SEQ ID NO: 28 - protein, 15 aa, synthetic construct
ESKSCDTPPPCPRCP

SEQ ID NO: 29 - protein, 13 aa, synthetic construct
ESKYGPPPCPRCP

SEQ ID NO: 30 - protein, 12 aa, synthetic construct
ESKYGPPCPPCP

SEQ ID NO: 31 - protein, 15 aa, synthetic construct
EXKSCDTPPPXPXCP

SEQ ID NO: 32 - protein, 28 aa, synthetic construct
MFWVLVVVGGVLACYSLLVTVAFIIFWV

SEQ ID NO: 33 - protein, 42 aa, synthetic construct
KRGRKKLLYIFKQPFMRPVQTTQEEDGCSCRFPEEEEGGCEL

SEQ ID NO: 34 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 442 aa, synthetic construct
DIQMTQTTSSLSASLGDRVTISCRASQDISKYLNWYQQRPDGTVKLLIYHTSRLHSGVPSRFSGSGSGTDYSLTISNLEPEDIATYFCQQGNTLPYTFGSGTRLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 442 aa, synthetic construct
DIQMTQTTSSLSASLGDRVTISCRASQDISKYLNWYQQRPDGTVKLLIYHTSRLHSGVPSRFSGSGSGTDYSLTISNLEPEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 442 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 22 aa, synthetic construct
MLLLVTSLLLCELPHPAFLLIP

SEQ ID NO: 39 - protein, 20 aa, synthetic construct
METDTLLLWVLLLWVPGSTG

SEQ ID NO: 40 - protein, 357 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 41 - protein, 18 aa, synthetic construct
EGRGSLLTCGDVEENPGP

SEQ ID NO: 42 - protein, 5 aa, synthetic construct
  REPEAT 1..5: note = Repeated for 1, 2, 3, 4, 5, 6, 7 or 8, in particular 3 times
GGGGS

SEQ ID NO: 43 - protein, 5 aa, synthetic construct
  REPEAT 1..5: note = Repeated for 1, 2, 3, 4, 5, 6, 7 or 8, in particular 3 times
SGGGG

SEQ ID NO: 44 - protein, 9 aa, synthetic construct
  REPEAT 5..9: note = Repeated for 1, 2, 3, 4, 5, 6, 7 or 8, in particular 3 times
GGGGSGGGG